SEQUENCE LISTING (22 sequences)

SEQ ID NO: 1 - protein, 5 aa, Artificial Sequence
  The sequence is synthesized
YGFGG

SEQ ID NO: 2 - protein, 5 aa, Artificial Sequence
  The sequence is synthesized
YRFGG

SEQ ID NO: 3 - protein, 6 aa, Artificial Sequence
  The sequence is synthesized
YRGFGG

SEQ ID NO: 4 - protein, 5 aa, Artificial Sequence
  The sequence is synthesized
YPFGG

SEQ ID NO: 5 - protein, 5 aa, Artificial Sequence
  The sequence is synthesized
GGFGY

SEQ ID NO: 6 - protein, 5 aa, Artificial Sequence
  The sequence is synthesized
GGFRY

SEQ ID NO: 7 - protein, 6 aa, Artificial Sequence
  The sequence is synthesized
GGFGRY

SEQ ID NO: 8 - protein, 5 aa, Artificial Sequence
  The sequence is synthesized
GGFPY

SEQ ID NO: 9 - protein, 39 aa, Artificial Sequence
  The sequence is synthesized
  MOD_RES (29)..(29): Ala is Ala(N-Me)
AVSEHQLLHDKGKSIQDLRRRELLEKLLAKLHTAYGFGG

SEQ ID NO: 10 - protein, 39 aa, Artificial Sequence
  The sequence is synthesized
  UNSURE (29): Xaa is Aib
AVSEHQLLHDKGKSIQDLRRRELLEKLLXKLHTAYGFGG

SEQ ID NO: 11 - protein, 40 aa, Artificial Sequence
  The sequence is synthesized
AVSEHQLLHDKGKSIQDLRRRELLEKLLAKLHTAYRGFGG

SEQ ID NO: 12 - protein, 39 aa, Artificial Sequence
  The sequence is synthesized
AVSEHQLLHDKGKSIQDLRRRFFLHHLIAEIHTAYGFGG

SEQ ID NO: 13 - protein, 39 aa, Artificial Sequence
  The sequence is synthesized
  UNSURE (29): Xaa is Aib
AVSEHQLLHDKGKSIQDLRRRFFLHHLIXEIHTAYRFGG

SEQ ID NO: 14 - protein, 39 aa, Artificial Sequence
  The sequence is synthesized
  UNSURE (29): Xaa is Aib
AVSEHQLIHDKGKSIQELRRRFFLHHLIXEIHTAYGFGG

SEQ ID NO: 15 - protein, 39 aa, Artificial Sequence
  The sequence is synthesized
AVSEHQLIHDKGKSIQELRRRFFLHHLLAEIHTAYGFGG

SEQ ID NO: 16 - protein, 34 aa, Artificial Sequence
  The sequence is synthesized
  MOD_RES (29)..(29): Ala is Ala(N-Me)
AVSEHQLLHDKGKSIQDLRRRELLEKLLAKLHTA

SEQ ID NO: 17 - protein, 34 aa, Artificial Sequence
  The sequence is synthesized
  UNSURE (29): Xaa is Aib
AVSEHQLLHDKGKSIQDLRRRELLEKLLXKLHTA

SEQ ID NO: 18 - protein, 34 aa, Artificial Sequence; identical to SEQ ID NO: 16
  The sequence is synthesized

SEQ ID NO: 19 - protein, 34 aa, Artificial Sequence
  The sequence is synthesized
AVSEHQLLHDKGKSIQDLRRRFFLHHLIAEIHTA

SEQ ID NO: 20 - protein, 34 aa, Artificial Sequence
  The sequence is synthesized
  UNSURE (29): Xaa is Aib
AVSEHQLLHDKGKSIQDLRRRFFLHHLIXEIHTA

SEQ ID NO: 21 - protein, 34 aa, Artificial Sequence
  The sequence is synthesized
  UNSURE (29): Xaa is Aib
AVSEHQLIHDKGKSIQELRRRFFLHHLIXEIHTA

SEQ ID NO: 22 - protein, 34 aa, Artificial Sequence
  The sequence is synthesized
AVSEHQLIHDKGKSIQELRRRFFLHHLLAEIHTA

(19) United States
(12) Patent Application Publication
(10) Pub. No.: US 2021/0107953 A1
(43) Pub. Date: Apr. 15, 2021

(54) ACTIVE POLYPEPTIDE COMPOUND

(71) Applicant: SHAANXI MICOT TECHNOLOGY LIMITED COMPANY, Xi'an (CN)

(72) Inventor: Guoqin FU, Xi'an (CN)

(21) Appl. No.: 16/727,078

(22) Filed: Dec. 26, 2019

(30) Foreign Application Priority Data
Oct. 10, 2019 (CN) 201910958680.0

(51) Int. Cl. C07K 14/435; A61P 19/10

(52) U.S. Cl. CPC C07K 14/435; A61K 45/06; A61P 19/10

(57) ABSTRACT

The present disclosure relates to the field of drug technology, specifically to an active polypeptide compound, which is Y-ID-X or X-ID-Y; wherein Y is a PTH/PTHrP receptor agonist or an osteoclast inhibitor; ID is a peptide bond or a linker in the molecule, which links X to Y; and X is an osteogenic growth peptide receptor agonist, a bone marrow mesenchymal stem cell irritant or a hematopoietic stem cell irritant. The present disclosure also relates to a pharmaceutical composition comprising the compound, and use of the compound and the pharmaceutical composition in the preparation of a medicament for preventing, treating or alleviating diseases or disorders related to osteogenic defects or bone mineral density decreasing.

### CROSS REFERENCE TO RELATED APPLICATIONS

This application claims the priority of Chinese Patent Application No. 201910958680.0, filed on Oct. 10, 2019, and titled with “ACTIVE POLYPEPTIDE COMPOUND”, and the disclosures of which are hereby incorporated by reference.

### FIELD

The present disclosure relates to the field of drug technology, specifically to a compound that can effectively promote ossification, and a pharmaceutical composition comprising the compound. Especially, the compound according to the present disclosure is a bispecific fusion polypeptide compound.

### BACKGROUND

Osteoporosis is a metabolic bone disease that is characterized by a decrease in bone mass and destruction of the microstructure of bone tissue, leading to increased bone pain and bone fragility, and prone to fracture. At present, there are more than 1.02 billion people with osteoporosis worldwide, and this number is expected to rise to 1.36 billion by 2030. The number of patients with fractures caused by osteoporosis will also reach 289,000, and the economic burden caused thereby will reach several billions every year. The situation in China is also not optimistic. A 2016 study using 2.0 SD as the diagnostic standard found that the osteoporosis population over the age of 40 nationwide was about 140 million, accounting for 24.62% of the total population.

As the deepening of understanding of osteoporosis, osteoporosis is thought to be caused by a variety of causes, which are generally three types: the first is primary osteoporosis, which is a physiological degenerative disease that occurs with age; the second is secondary osteoporosis, which is induced by other diseases or drugs or other factors; the third is idiopathic osteoporosis, which is often found in adolescents aged 8-14, most of which have a family genetic history, and the female are more than the male. Primary osteoporosis can be divided into two types. Type I is postmenopausal osteoporosis, which is a highly conversion type osteoporosis. Type II is senile osteoporosis, which is a low conversion type, and usually occurs in the senior over 65 years old. The pathogenesis of postmenopausal osteoporosis is relatively simple, which mainly relates to increase of osteoclast function caused by lack of estrogen and accelerated bone loss caused by promoting bone resorption; in addition, bone loss increases with age, bone mass decreases and bone mineral density is far below the peak, and prolonged staying in bed accelerates bone loss. The main clinical manifestations of osteoporosis are: \textcircled{1} pain, which is manifested as low back pain or skeletal pain around the body, and the pain worsens when the load increases, and when the pain is serious, it is hard to turn over, get up and walk; \textcircled{2} spine deformity: shortened height or hump, spine deformity or restricted stretch. \textcircled{3} fracture.

With the prolongation of human life and the aging of society, osteoporosis has become an important health problem of human beings. At present, China has a population of about 173 million people over the age of 60, making it the country with the largest absolute number of elderly people in the world. A large-scale national epidemiological survey from 2003 to 2006 showed that the overall prevalence of osteoporosis over the age of 50 based on bone mineral density values of verteb and femoral bone and neck bone was 20.7% in women and 14.4% in men. The prevalence of osteoporosis is significantly increased in people over 60 years of age, especially in women. Estimated by survey, in 2006, about 69.44 million people over 50 years old suffered from osteoporosis, and about 210 million people had low bone mass. It is estimated that the hip fracture rate of Chinese people will increase significantly in the next few decades. In a woman's lifetime, the risk of osteoporotic fractures (40%) is higher than the sum of breast cancer, endometrial cancer, and ovarian cancer.

Osteoporosis is not caused by a single factor. Factors involved in the pathogenesis include: \textcircled{1} genetic factors; \textcircled{2} deficiency of calcium and vitamin D; \textcircled{3} osteoporosis caused by insufficient estrogen, and the effect of estrogen replacement has been widely recognized; \textcircled{4} androgen deficiency is also involved in male osteoporosis; and \textcircled{5} degenerative mechanism of old age.

Osteoporosis should be treated as early as possible. Although the completely and partially disappeared bone units (columnar bone unit and trabeculae with a diameter of 0.2 mm of cortical bone) cannot be regenerated, the thinned bone units can be restored to their original state after treatment. Therefore, it is impossible to reverse the disappeared bone units (formation of osteoporosis), but early intervention can prevent osteoporosis of most people. Perimenopause women (45 years old) should start treatment, and men can start ten years later.

The drugs used to treat and prevent the development of osteoporosis are divided into three categories. The first category is a drug that inhibits bone resorption, such as calcitonin, diphosphates, estrogen, and isoflavone. The second category is a drug that promotes ossification, including fluoride, synthesized steroid, parathyroid hormone and isoflavone. The third category is a drug that promotes bone mineralization, including calcium agents, vitamin D and active vitamin D. Therein, the anti-osteoporosis treatment drugs are mainly calcitonin, bone calcium regulators, selective estrogen receptor modulators (SERMS) and parathyroid hormones (PTHs). Estrogen can cause the risk of breast cancer and endometrial cancer, and calcitonin easily causes hyperparathyroidism and produce antibodies. Selective estrogen receptor modulators (SERMs), such as raloxifene, can reduce the incidence of new spinal fractures (a 30-50% decrease in spinal fractures), but its effect on hip and other non-vertebral fractures is unclear. Bisphosphonate drugs have poor bioavailability, and must be taken on an empty stomach with water and kept at least 30 minutes in the non-recumbent position and without food, bringing about a lot of inconvenience to patients. Parathyroid hormone (PTH) is also used as a ossification promoter in the treatment of post-menopausal osteoporosis with high-risk fractures to increase bone mineral density, bone markers, and reduce the risk of fractures. Parathyroid hormone is also approved for primary or hypogonadal osteoporosis in men with high-risk fractures. Studies have confirmed that parathyroid hormone can reduce new spine cases by 65%-69%. Parathyroid hormone analogues, teriparatide as a currently marketed drug of this type, stimulates ossification and bone resorption, which can reduce the incidence of fractures in postmenopausal women. Depending on the mode of administration, it can also increase or decrease the bone mineral density. The common adverse reactions of teriparatide are nausea, limb pain, dizziness and swirl. However, it is worth noting that if there is an increased risk of osteosarcoma, parathyroid hormone should not be used. For children, patients with unclosed metaphysis, patients with tumor bone metastasis or bone malignancy, patients with metabolic bone disease other than osteoporosis, patients with existing hypercalcemia, or patients who have previously experienced bone radiation therapy, parathyroid hormone should not be used.

RANKL inhibitor drug, for example, denosumab, is a human-derived IgG2 monoclonal antibody, which can inhibit the formation, activation and survival of osteoclast by specific binding with RANKL, so as to reduce the incidence of fractures. Denosumab, as a specific RANKL inhibitor, opens a new mechanism for anti-bone resorption. Beaudoin et al. found that after 12 to 24 months of treatment, there was no significant difference between denosumab and bisphosphonates in reducing the risk of fracture. However, since the OPG/RANK/RANKL signaling pathway also participates in the human immune response, the drug, as an inhibitor of this pathway, may have the risk of causing immune diseases, and the safety of long-term application needs further research.

In addition, parathyroid hormone-related protein (PTHrP), also referred to as parathyroid hormone-like hormone (PTHLH), shares many biological effects with PTH, including binding to a common PTH/PTHrP receptor. At present, parathyroid hormone-related protein analog drugs have gradually become one of the new research directions for osteoporosis drugs.

In view of the current situation, great progress has been made in the treatment of osteoporosis, but it is still not possible to completely and continuously correct the decline of bone mineral density and osteoporosis. The condition of the majority of osteoporosis patients has not been controlled in a timely and effective manner, and the treatment of osteoporosis has not reached the ideal goal, and further research is needed. At present, the continuous development of peptide compounds that promote osteogenesis with better efficacy and fewer side effects is of great significance for the treatment of osteoporosis and the treatment and prevention of osteoporotic fractures.

### SUMMARY

In one aspect, one object of the present disclosure is providing an active polypeptide compound, and the active polypeptide compound provided by the present disclosure has multi-target activity, and can play a role of regulation or treatment in multiple aspects at the same time.

In order to realize the above object of the disclosure, the present disclosure adopts the following technical solution.

The present disclosure firstly provides an active polypeptide compound, which has a structure represented by following Formula (Ia) or Formula (Ib), or is a pharmaceutically acceptable salt thereof,

Y-ID-X Formula (Ia), or

X-ID-Y Formula (Ib),

wherein,

Y is a PTH/PTHrP receptor agonist or an osteoclast inhibitor;

ID is a peptide bond or a linker in the molecule, which links X to Y; and

X is an osteogenic growth peptide receptor agonist, a bone marrow mesenchymal stem cell irritant or a hematopoietic stem cell irritant.

For the active polypeptide compound Y-ID-X (Ia) or X-ID-Y (Ib) in the present disclosure, on the one hand, some compounds can exert dual-action activities as a whole, by exerting different physiological effects at different active regions; on the other hand, ID structure in the molecules of some compounds decomposes in body. When ID is a peptide bond, it breaks via hydrolyzation, giving polypeptide Y (as the PTH/PTHrP receptor agonist or osteoclast inhibitor) and polypeptide X (as the osteogenic growth peptide receptor agonist, bone marrow mesenchymal stem cell irritant or hematopoietic stem cell irritant), and they function respectively. Alternatively, when ID is a linker, ID can also release active peptide X and active peptide Y via hydrolyzation or enzymolysis. In the present disclosure, the active polypeptide compound can not only bind with the PTH/PTHrP receptors and stimulate PTH/PTHrP receptors or inhibit osteoclast, so as to promote ossification and increase bone mineral density, but also play a role of maintaining the karyocyte level (for example mononuclear cell, lymphocyte and white blood cell) in peripheral blood by stimulating osteogenic growth peptide receptor and stimulating hematopoietic stem cells.

In some embodiments, Yin the active polypeptide compound of the present disclosure is an M-CSF (macrophage colony-stimulating factor, also known as colony-stimulating factor-1) antagonist, an RANKL (receptor activator of nuclear factor κ B ligand) inhibitor, an RANKL antibody, an MMP (matrix metalloproteinase) inhibitor, calcitonin, parathyroid hormone or parathyroid hormone-related protein.

In some embodiments, Yin the active polypeptide compound of the present disclosure is a peptide chain having an amino acid sequence as shown in Formula (II):

A₁-Val-Ser-Glu-His-Gln-Leu-A₈-His-Asp-Lys-Gly-Lys-Ser-Ile-Gln-A₁₇-Leu-Arg-Arg-Arg-A₂₂-A₂₃-Leu-A₂₅-A₂₆-Leu-A₂₈-A₂₉-A₃₀-A₃₁-His-Thr-Ala Formula (II);

wherein, A₁ is Ala, Val, Leu or Ile;

A₈ is Leu or Ile;

A₁₇ is Asp or Glu;

A₂₂ is Glu, Asp or Phe;

A₂₃ is Leu, Ile or Phe;

A₂₅ is Glu, Asp or His;

A₂₆ is Lys, His or Arg;

A₂₈ is Leu, Ile or Val;

A₂₉ is Ala, (N-Me)Ala or Aib;

A₃₀ is Lys or Glu;

A₃₁ is Leu or Ile;

the amino terminal of the peptide chain Y is free or chemically modified, and the carboxyl terminal of the peptide chain Y is free or chemically modified.

In specific embodiments of the present disclosure, in the active polypeptide compounds of the present disclosure, the amino acids in the peptide chain Y are all L-type amino acids.

In some specific embodiments of the present disclosure, in the active polypeptide compound of the present disclosure, Y is one of the polypeptides having a structure as shown in the following SEQ ID No.16-SEQ ID No.22:

(1)
SEQ ID NO: 16
Ala-Val-Ser-Glu-His-Gln-Leu-Leu-His-Asp-Lys-Gly-
Lys-Ser-Ile-Gln-Asp-Leu-Arg-Arg-Arg-Glu-Leu-Leu-
Glu-Lys-Leu-Leu-(N-Me)Ala-Lys-Leu-His-Thr-Ala;
(2)
SEQ ID NO: 17
Ala-Val-Ser-Glu-His-Glu-Leu-Leu-His-Asp-Lys-Gly-
Lys-Ser-Ile-Gln-Asp-Leu-Arg-Arg-Arg-Glu-Leu-Leu-
Glu-Lys-Leu-Leu-Aib-Lys-Leu-His-Thr-Ala;
(3)
SEQ ID NO: 18
Ala-Val-Ser-Glu-His-Gln-Leu-Leu-His-Asp-Lys-Gly-
Lys-Ser-Ile-Gln-Asp-Leu-Arg-Arg-Arg-Glu-Leu-Leu-
Glu-Lys-Leu-Leu-Ala-Lys-Leu-His-Thr-Ala;
(4)
SEQ ID NO: 19
Ala-Val-Ser-Glu-His-Gln-Leu-Leu-His-Asp-Lys-Gly-
Lys-Ser-Ile-Gln-Asp-Leu-Arg-Arg-Arg-Phe-Phe-Leu-
His-His-Leu-Ile-Ala-Glu-Ile-His-Thr-Ala;
(5)
SEQ ID NO: 20
Ala-Val-Ser-Glu-His-Gln-Leu-Leu-His-Asp-Lys-Gly-
Lys-Ser-Ile-Gln-Asp-Leu-Arg-Arg-Arg-Phe-Phe-Leu-
His-His-Leu-Ile-Aib-Glu-Ile-His-Thr-Ala;
(6)
SEQ ID NO: 21
Ala-Val-Ser-Glu-His-Gln-Leu-Ile-His-Asp-Lys-Gly-
Lys-Ser-Ile-Gln-Glu-Leu-Arg-Arg-Arg-Phe-Phe-Leu-
His-His-Leu-Ile-Aib-Glu-Ile-His-Thr-Ala;
(7)
SEQ ID NO: 22
Ala-Val-Ser-Glu-His-Gln-Leu-Ile-His-Asp-Lys-Gly-
Lys-Ser-Ile-Gln-Glu-Leu-Arg-Arg-Arg-Phe-Phe-Leu-
His-His-Leu-Leu-Ala-Glu-Ile-His-Thr-Ala

In some embodiments, X in the active polypeptide compound of the present disclosure is a hematopoietic stem cell irritant, and that is to say, X is a hematopoietic growth factor, a platelet colony-stimulating factor, a granulocyte colony-stimulating factor, erythropoietin, interleukin 3 (IL3) or recombinant human interleukin 11.

In some embodiments, X in the active polypeptide compound of the present disclosure is a peptide chain having an amino acid sequence as shown in Formula (IIIa) or Formula (IIIb):

Tyr-(Arg)ₘ-(Gly)ₙ-Phe-Gly-Gly Formula (IIIa)

Gly-Gly-Phe-(Gly)ₙ-(Arg)ₘ-Tyr Formula (IIIb);

wherein, m and n are independently 0, 1 or 2; and

the amino terminal of the peptide chain X is free or chemically modified, and the carboxyl terminal of the peptide chain X is free or chemically modified.

In some embodiments, X in the active polypeptide compound of the present disclosure is a peptide chain consisting of 5-6 amino acids, which has an amino acid sequence as shown in the following SEQ ID NO:1-SEQ ID NO:8:

(SEQ ID NO: 1)
Tyr-Gly-Phe-Gly-Gly
(SEQ ID NO: 2)
Tyr-Arg-Phe-Gly-Gly
(SEQ ID NO: 3)
Tyr-Arg-Gly-Phe-Gly-Gly
(SEQ ID NO: 4)
Tyr-Pro-Phe-Gly-Gly
(SEQ ID NO: 5)
Gly-Gly-Phe-Gly-Tyr
(SEQ ID NO: 6)
Gly-Gly-Phe-Arg-Tyr
(SEQ ID NO: 7)
Gly-Gly-Phe-Gly-Arg-Tyr
(SEQ ID NO: 8)
Gly-Gly-Phe-Pro-Tyr.

In some embodiments, ID in the active polypeptide compound of the present disclosure is a linker between X and Y; the linker is an amino-substituted C₁₋₈ alkyl acid, a polyethylene glycol polymer chain or a peptide segment consisting of 1-10 amino acids, and the amino acids in the peptide segment is selected from the group consisting of proline, arginine, alanine, threonine, glutamic acid, aspartic acid, lysine, glutamine, asparagine and glycine.

In some specific embodiments of the present disclosure, the linker is one of the following linkers:

(1) (Gly-Ser)ₚ, wherein p is 1, 2, 3, 4 or 5;

(2) (Gly-Gly-Gly-Gly-Ser)ₜ, wherein t is 1, 2 or 3;

(3) Ala-Glu-Ala-Ala-Ala-Lys-Ala;

(4) 4-aminobutyric acid or 6-aminocaproic acid; and

(5) (PEG)_{q}, wherein q is 1, 2, 3, 4 or 5.

In some embodiments, the active polypeptide compound according to the present disclosure has a structure as shown in Formula (IV), or is a pharmaceutically acceptable salt of the compound shown as in Formula (IV):

A₁-Val-Ser-Glu-His-Gln-Leu-A₈-His-Asp-Lys-Gly-Lys-Ser-Ile-Gln-A₁₇-Leu-Arg-Arg-Arg-A₂₂-A₂₃-Leu-A₂₅-A₂₆-Leu-A₂₈-A₂₉-A₃₀-A₃₁-His-Thr-Ala-A₃₅ Formula (IV),

wherein,

A₁ is Ala, Val, Leu or Ile;

A₈ is Leu or Ile;

A₁₇ is Asp or Glu;

A₂₂ is Glu, Asp or Phe;

A₂₃ is Leu, Ile or Phe;

A₂₅ is Glu, Asp or His;

A₂₆ is Lys, His or Arg;

A₂₈ is Leu, Ile or Val;

A₂₉ is Ala, (N-Me)Ala or Aib;

A₃₀ is Lys or Glu;

A₃₁ is Leu or Ile; and

A₃₅ has a peptide chain of the amino acid sequence as shown in Formula (IIIa) or (IIIb):

Tyr-(Arg)ₘ-(Gly)ₙ-Phe-Gly-Gly Formula (IIIa),

Gly-Gly-Phe-(Gly)ₙ-(Arg)ₘ-Tyr Formula (IIIb),

wherein, m and n are independently 0, 1 or 2; and

the amino terminal of the amino acids shown by A₁ is free or chemically modified, and the carboxyl terminal of the peptide chain A₃₅ is free or chemically modified.

In some embodiments, in the active polypeptide compound of the present disclosure, the peptide can be modified on N-terminal (amino terminal), C-terminal (carboxyl terminal) or both terminals. The chemical modifications of the amino terminal include acylation, sulfonylation, alkylation and PEG modification; and the chemical modifications of the carboxyl terminal include amidation, sulfonylation and PEG modification.

Further, the chemical modification of the amino terminal is acetylation, benzoylation or sulfonylation of amino; the alkylation of amino terminal is C₁₋₆ alkylation or aromatic alkylation; the chemical modification of carboxylic terminal is that the OH in the carboxyl is substituted by NH₂ or sulfamide, or the OH in the carboxyl links to a functionalized PEG molecule.

In some specific embodiments of the present disclosure, the compound as shown in Formula (Ia) or Formula (Ib) is a compound of one of the following SEQ ID NO:9-SEQ ID NO:15, or a pharmaceutically acceptable salt thereof:

(1)
SEQ ID NO: 9
Ala-Val-Ser-Glu-His-Gln-Leu-Leu-His-Asp-Lys-Gly-
Lys-Ser-Ile-Gln-Asp-Leu-Arg-Arg-Arg-Glu-Leu-Leu-
Glu-Lys-Leu-Leu-(N-Me)Ala-Lys-Leu-His-Thr-Ala-
Tyr-Gly-Phe-Gly-Gly;
(2)
SEQ ID NO: 10
Ala-Val-Ser-Glu-His-Gln-Leu-Leu-His-Asp-Lys-Gly-
Lys-Ser-Ile-Gln-Asp-Leu-Arg-Arg-Arg-Glu-Leu-Leu-
Glu-Lys-Leu-Leu-Aib-Lys-Leu-His-Thr-Ala-Tyr-Gly-
Phe-Gly-Gly;
(3)
SEQ ID NO: 11
Ala-Val-Ser-Glu-His-Gln-Leu-Leu-His-Asp-Lys-Gly-
Lys-Ser-Ile-Gln-Asp-Leu-Arg-Arg-Arg-Glu-Leu-Leu-
Glu-Lys-Leu-Leu-Ala-Lys-Leu-His-Thr-Ala-Tyr-Arg-
Gly-Phe-Gly-Gly;
(4)
SEQ ID NO: 12
Ala-Val-Ser-Glu-His-Gln-Leu-Leu-His-Asp-Lys-Gly-
Lys-Ser-Ile-Gln-Asp-Leu-Arg-Arg-Arg-Phe-Phe-Leu-
His-His-Leu-Ile-Ala-Glu-Ile-His-Thr-Ala-Tyr-Gly-
Phe-Gly-Gly;
(5)
SEQ ID NO: 13
Ala-Val-Ser-Glu-His-Gln-Leu-Leu-His-Asp-Lys-Gly-
Lys-Ser-Ile-Gln-Asp-Leu-Arg-Arg-Arg-Phe-Phe-Leu-
His-His-Leu-Ile-Aib-Glu-Ile-His-Thr-Ala-Tyr-Arg-
Phe-Gly-Gly;
(6)
SEQ ID NO: 14
Ala-Val-Ser-Glu-His-Gln-Leu-Ile-His-Asp-Lys-Gly-
Lys-Ser-Ile-Gln-Glu-Leu-Arg-Arg-Arg-Phe-Phe-Leu-
His-His-Leu-Ile-Aib-Glu-Ile-His-Thr-Ala-Tyr-Gly-
Phe-Gly-Gly;
(7)
SEQ ID NO: 15
Ala-Val-Ser-Glu-His-Gln-Leu-Ile-His-Asp-Lys-Gly-
Lys-Ser-Ile-Gln-Glu-Leu-Arg-Arg-Arg-Phe-Phe-Leu-
His-His-Leu-Leu-Ala-Glu-Ile-His-Thr-Ala-Tyr-Gly-
Phe-Gly-Gly

Further, the active polypeptide compound of the present disclosure further includes a compound obtained by chemically modifying the side chain groups of amino acids of the polypeptide compound; or

a coordination compound, a complex or a chelate formed by the polypeptide compound and a metal ion; or

a hydrate or a solvate formed by the polypeptide compound.

In some embodiments, the compound obtained by chemically modifying the side chain groups of amino acids of the polypeptide compound is a thioether or thioglycoside formed from a sulfydryl in the cysteine in the polypeptide compound, or a compound having a disulfide bond formed from a cysteine or a peptide comprising cysteine; or

an ester, an ether and a glycoside formed from a phenolic hydroxyl group of a tyrosine in the polypeptide compound; or

a compound prepared by substituting a benzene ring of a tyrosine or phenylalanine in the polypeptide compounds.

It should be noted that other variants of the polypeptide compound disclosed in the present disclosure are also included in the scope of the present disclosure, especially including any variants obtained by merely replacing the conserved amino acids.

The active polypeptide compound provided by the present disclosure can exist in the form of a free polypeptide or a salt. In some embodiments, the salt is a pharmaceutically acceptable salt.

Term “pharmaceutically acceptable” means that a substance or composition must be chemically and/or toxicologically compatible with other components contained in the preparation and/or the mammal to be treated.

In the present disclosure, the “pharmaceutically acceptable salt” may be prepared by a parent compound and an alkaline or acid part by a routine chemical method. Generally speaking, such a salt can be prepared by reacting the free acid form of such a compound with a stoichiometric amount of a suitable base (e.g. hydroxide, carbonate, bicarbonate, etc. of Na, Ca, Mg or K), or by reacting the free base form of such a compound with a stoichiometric amount of a suitable acid, these salts. This kind of reaction usually carries out in water, an organic solvent or a mixture thereof. Generally, in suitable conditions, a non-aqueous medium such as ethyl ether, ethyl acetate, ethanol, isopropanol or acetonitrile is needed to be used. Examples of the salt include, but not limited to organic acids (e.g. acetic acid, trifluoroacetic acid, lactic acid, maleic acid, citric acid, malic acid, ascorbic acid, succinic acid, benzoic acid, methanesulfonic acid, toluenesulfonic acid or pamoic acid), inorganic acids (e.g. hydrochloric acid, sulfuric acid or phosphoric acid) and polymeric acids (e.g. tannic acid, carboxymethyl cellulose, polylactic acid, polyglycolic acid or polylactic acid-glycolic acid copolymer). In, for example, “Remington's Pharmaceutical Sciences”, the 20^{th} Edition, Mack Publishing Company, Easton, Pa., (1985); and (Handbook of Pharmaceutical Salts: Properties, Selection, and Use)”, Stahl and Wermuth (Wiley-VCH, Weinheim, Germany, 2002), a list of other suitable salts can be found.

The active polypeptide compound provided by the present disclosure is constructed by linking structures, and belongs to an active polypeptide having multi-target effects, which can not only stimulate a PTH/PTHrP receptor or inhibit osteoclast, but also stimulate osteogenic growth peptide receptor, stimulate bone marrow mesenchymal stem cell or stimulate hematopoietic stem cell. The active polypeptide compound provided by the present disclosure stimulates PTH/PTHrP receptor. On the one side, the active polypeptide compound, by influencing multiple cell lines (e.g., osteoblast, osteoclast, bone lining cell, bone cell, etc.), stimulats AC-Camp-PKA pathway, and plays a role of facilitating ossification (bone and cartilage) such as increasing osteoblast activity, increasing bone mass, increasing bone mineral density and improving bone strength and the like; on the other side, the polypeptide compound provided by the present disclosure has effects on bone marrow mesenchymal stem cell and promotes its differentiation towards osteoblast, including increasing the activity of ALP, up-regulating type I collagen, osteocalcin and transcription of Cbfα1 mRNA, promoting calcium salt deposition and matrix mineralization, promoting ossification, accelerating fracture healing, increasing bone mineral density, etc. It acts on bone marrow hematopoietic stem cells and improves hematopoietic microenvironment of marrow by up-regulating osteoblasts and other bone marrow cell lines to produce hematopoietic stimulating factors.

The pharmacodynamics activity experiments of the present disclosure demonstrates that the polypeptide compound provided by the present disclosure can significantly increase the bone mineral density of lumbar vertebra and thigh bone of ovary removed osteoporosis model rats. The increased percentages of bone mineral density of lumbar vertebra and bone mineral density of thigh bone of administration groups of active polypeptide compounds in the present disclosure are comparative to that of the positive control abaloptide group. During the period administering abaloptide, there was significant inhibition effect on peripheral karyocyte such as mononuclear cell, lymphocyte and white blood cells, but the active polypeptide compound of the present disclosure does not have adverse effects on peripheral blood karyocyte, showing that the active polypeptide compound of the present disclosure overcomes the adverse effects of abaloptide, avoiding the influence on immunity during the period of administering.

The pharmacodynamics activity experiments of the present disclosure further demonstrates that the active polypeptide compound of the present disclosure can increase the peak load of thigh bone of retinoic acid-induced osteoporosis rat, improve microstructure of bone, specifically including improving bone surface area/bone volume ratio, trabeculae number (TbN) and trabecular spacing, and the effect is better than the marketed drug abaloptide; and the active polypeptide compound of the present disclosure can avoid the adverse effect of bone marrow inhibition caused by abaloptide.

On this basis, the present disclosure further provides a pharmaceutical composition, comprising the active polypeptide compound of the present disclosure. Optionally, the pharmaceutical composition further comprises at least one of a pharmaceutically acceptable adjuvant, excipient, carrier and solvent thereof.

In some embodiments, the pharmaceutical composition of the present disclosure further comprises other therapeutic agents. The other therapeutic agents are selected from a drug that inhibits bone resorption, a drug that promotes ossification, a drug that promotes bone mineralization or parathyroid hormone-related protein.

Therein, the drug that inhibits bone resorption includes calcitonin, diphosphonate, oestrogen, selective oestrogen receptor regulators and isoflavone; the drug that promotes ossification includes fluoride, synthesized steroid, parathyroid hormone and parathyroid hormone-related protein; the drug that promotes bone mineralization includes calcium agents, vitamin D and active vitamin D; and the parathyroid hormone-related protein is teriparatide or abaloptide.

In the present disclosure, “pharmaceutically acceptable adjuvant” means a pharmaceutically acceptable material, mixture, or solvent that is related to the consistency of the dosage form or pharmaceutical composition. Each adjuvant must be compatible with other components of the pharmaceutical composition when mixed, so as to avoid interactions that greatly reduce the efficacy of the active polypeptide compound disclosed herein and interactions that lead to non-pharmaceutically acceptable pharmaceutical composition when administered to a patient. In addition, each adjuvant must be pharmaceutically acceptable, for example, with a sufficiently high purity. Suitable pharmaceutically acceptable adjuvant varies depending on the particular dosage form chosen. In addition, pharmaceutically acceptable adjuvant can be selected based on their specific function in the composition. For example, some pharmaceutically acceptable accessories that facilitate producing a uniform dosage form can be selected. Certain pharmaceutically acceptable adjuvant s that facilitate producing stable dosage forms can be selected. Certain pharmaceutically acceptable adjuvants that facilitate carrying or conveying the active polypeptide compound disclosed herein from one organ or part of body to another organ or part of body when administered to a patient can be selected. Certain pharmaceutically acceptable adjuvants that enhance patient compliance can be selected. Suitable pharmaceutically acceptable adjuvants include the following types of adjuvants: diluents, fillers, binders, disintegrants, lubricants, glidants, granulating agents, coating agents, wetting agents, solvents, co-solvents, suspending agents, emulsifiers, sweeteners, flavoring agents, taste-masking agents, colorants, anti-caking agents, humectants, chelating agents, plasticizers, tackifiers, antioxidants, preservatives, stabilizers, surfactants and buffers. One of ordinary skill in the art will recognize that certain pharmaceutically acceptable adjuvants may provide more than one function and provide alternative functions, depending on how much of the adjuvant is present in the formulation and which other adjuvants are present in the formulation.

In another aspect, the present disclosure further provides use of the active polypeptide compound of the present disclosure and the pharmaceutical composition in preparation of a medicament for preventing, treating or alleviating diseases or disorders related to osteogenic defects and bone mineral density decreasing, and the diseases include osteoporosis.

In another aspect, the present disclosure further provides use of the active polypeptide compound of the present disclosure and the pharmaceutical composition in preparation of a medicament, which is used in stimulating PTH/PTHrP receptor, inhibiting osteoclast, stimulating osteogenic growth peptide receptor, stimulating bone marrow mesenchymal stem cell or hematopoietic stem cell.

The present disclosure further provides a method for preventing, treating or alleviating diseases or disorders related to osteogenic defects or bone mineral density decreasing, comprising administering an effective amount of the active polypeptide compound of the present disclosure or the pharmaceutical composition to a subject in need thereof.

The pharmaceutical compositions of the present disclosure can be used to stimulate the ossification of a subject. Thus, they can be used to treat diseases or obstacles related to osteogenic defects, such as osteoporosis.

In some embodiments, the present disclosure relates to a method of treating osteoporosis of a subject, comprising administering an effective amount of the pharmaceutical composition of the present disclosure to a subject.

Unless stated otherwise, all technical and scientific terms used herein have the same meaning as commonly understood by one of ordinary skill in the art to which this disclosure belongs. All patents and publications related to the present disclosure are incorporated herein by reference in their entirety.

In the present disclosure, the “amino acid” represents for natural and artificial amino acid. Twenty natural amino acids (L-isomer) are represented by three-letter codes or capital-letter one-letter codes. Unless particularly stated, the amino acids represented by three-letter codes are L-isomers, except for the achiral glycine: alanine (“Ala” or “A”), arginine (“Arg” or “R”), asparagine (“Asn” or “N”), aspartic acid (“Asp” or “D”), cysteine (“Cys” or “C”), glutamine (“Gln” or “Q”), glutamic acid (“Glu” or “E”), glycine (“Gly” or “G”), histidine (“His” or “H”), isoleucine (“Ile” or “I”), leucine (“Leu” or “L”), lysine (“Lys” or “K”), methionine (“Met” or “M”), phenylalanine (“Phe” or “F”), proline (“Pro” or “P”), serine (“Ser” or “S”), threonine (“Thr” or “T”), tryptophan (“Trp” or “W”), tyrosine (“Tyr” or “Y”) and valine (“Val” or “V”). L-norleucine and L-norvaline can be represented as (NLeu) and (NVal), respectively.

Nineteen natural chiral amino acids have corresponding D-isomer, and are represented by three-letter codes having a prefix “D-” or lowercase one-letter codes in the present disclosure: D-alanine (“D-Ala” or “a”), D-arginine (“D-Arg” or “r”), D-asparagine (“D-Asn” or “a”), D-aspartic acid (“D-Asp” or “d”), D-cysteine (“D-Cys” or “c”), D-type glutamine (“D-Gln” or “q”), D-glutamic acid (“D-Glu” or “e”), D-histidine (“D-His” or “h”), D-isoleucine (“D-Ile” or “i”), D-leucine (“D-Leu” or “l”), D-lysine (“D-Lys” or “k”), D-methionine (“D-Met” or “m”), D-phenylalanine (“D-Phe” or “f”), D-proline (“D-Pro” or “p”), D-serine (“D-Ser” or “s”), D-threonine (“D-Thr” or “t”), D-tryptophan (“D-Trp” or “w”), D-tyrosine (“D-Tyr” or “y”) and D-valine (“D-Val” or “v”).

Although “amino acid residues” is generally used to represent peptide, polypeptide or protein monomer subunits, and “amino acids” is generally used to represent free molecules, the terms “amino acid” and “amino acid residue” are used interchangeably in the present disclosure.

Every two amino acids are linked to each other to form a peptide bond, and multiple amino acids are linked to each other to form multiple peptide bonds. A chain structure containing multiple peptide bonds formed by the interconnection of multiple amino acids is called a “peptide chain” or “peptide segment.”

As used herein, “peptide” and “polypeptide” refer to polymers composed of amino acid residue chains connected by peptide bonds, regardless of their molecular size. The terms “peptide” and “polypeptide” are used interchangeably in the present disclosure.

Unless otherwise indicated, peptide sequences are given in an order from the amino-terminal (N-terminal) to the carboxyl-terminal (C-terminal).

PTH/PTHrP receptor located on osteoblasts (or stromal cell precursor) of PTH/PTHrP receptors agonist is a member of the G protein coupled receptor superfamily, which can be activated by the endogenous natural ligands PTH and PTHrP (1-36). Ligands bind to PTH/PTHrP receptors and can activate two signaling pathways in the cell. One is adenylate cyclase/cAMP/Gs-related protein kinase A. The other is inositol triphosphate/intracellular calcium/Gq-related protein kinase C pathway. PTH/PTHrP receptor agonists refer to substances that can bind to PTH/PTHrP receptor and activate intracellular signaling pathways, for example, but not limited to the peptide chain Y, PTHrP (1-36), PTH (1-34), teriparatide, etc., according to the present disclosure.

Osteoclast inhibitor: Osteoclast (OC) is the main functional cell of bone resorption, responsible for the dissolution of minerals and organic bone matrix. Osteoclast inhibitors can inhibit the formation or activity of osteoclasts, thereby blocking bone resorption. Osteoclast inhibitors include bisphosphonates (BP) that inhibit osteoclast activity and accelerate apoptosis, (such as alendronate, zometa, pyrophosphate analogs), M-CSF antagonists (such as M-CSF antibodies), RANKL inhibitors (such as RANKL antibodies), osteoprotegerin (OPG), platelet-derived growth factor (PDGF), and matrix metalloproteinase (MMP) inhibitor.

Osteogenic growth peptide receptor agonist (osteogenic growth peptide receptor activator) refer to a substance that can activates the osteogenic growth peptide receptor signaling pathway, promotes the proliferation and differentiation of osteoblasts, stimulates the proliferation of bone marrow hematopoietic stem cells and bone marrow mesenchymal stem cells, and can maintain the self-recovery ability of hematopoietic stem cells and inhibit the growth of megakaryocytes. Osteogenic growth peptide receptor agonists can be small molecule compounds or polypeptide molecules. Osteogenic growth peptide receptors are G protein-coupled receptors located on osteoblasts. Mitogen activated protein kinase (MAPK), Src and RhoA pathways can be activated after osteogenic growth peptide receptor agonist binds to osteogenic growth peptide receptor. Activation of the MAP pathway will increase mitosis and have a mitogenic proliferation effect on osteoblasts, bone marrow hematopoietic stem cells, and bone marrow mesenchymal stem cells; and activation of the Src and RhoA pathways can regulate the autocrine expression of endogenous osteogenic growth peptides of osteoblasts and promote alkaline phosphatase secretion, up-regulate the transcription of type I collagen, osteocalcin, and Cbfα1 mRNA, promote calcium salt deposition and matrix mineralization, promote osteogenesis, accelerate fracture healing, and increase bone mineral density. Osteogenic growth peptide receptor agonists include, but not limited to, immunoreactive OGP, specifically including free OGP, OGP (10-14), recombinant OGP and OCP-osteogenic growth peptide binding protein (OGPBP), as well as natural or artificial polypeptide compounds with similar activity, such as peptide chain X in the compounds of the present disclosure.

Bone marrow mesenchymal stem cell stimulants: Bone marrow mesenchymal stem cells are also known as bone marrow stromal cells. Bone marrow mesenchymal stem cells have mechanical support for hematopoietic stem cells (HSC) in the bone marrow, and can secrete a variety of cell factors (such as IL-6, IL-11, LIF, M-CSF and SCF, etc.) that regulate hematopoiesis to support hematopoiesis. They also have the potential for differentiation and can differentiate into osteoblasts, fibroblasts, reticulocytes, adipocytes and endothelial cells. Bone marrow mesenchymal stem cell irritant refers to substances that can stimulate bone marrow mesenchymal stem cells to secrete and regulate hematopoietic cell factors, thereby promoting hematopoietic function, and/or can induce bone marrow mesenchymal stem cells to proliferate and differentiate. Bone marrow mesenchymal stem cell irritants include, but not limited to, immunoreactive OGP, specifically including free OGP, OGP (10-14), recombinant OGP and OCP-osteogenic growth peptide binding protein (OGPBP), as well as natural or artificial polypeptide compounds with similar activity, such as peptide chain X in the compounds of the present disclosure.

Hematopoietic stem cell stimulants: Hematopoietic stem cells (HSCs) are a group of cells that have the abilities to self-renew and differentiate into all blood cells or immune cells. Hematopoietic stem cells can come from bone marrow, peripheral blood, and umbilical cord blood. An active substance that can stimulate hematopoietic stem cells and thereby promote hematopoiesis is called a hematopoietic stem cell irritant. The hematopoietic stem cell irritants of the present disclosure include hematopoietic growth factors (HGFs), platelet colony-stimulating factor, granulocyte colony-stimulating factor (G-CSF), erythropoietin (EPO), interleukin 3 (IL3), recombinant human interleukin 11 (IL11), TAT-H0XB4H recombinant protein, and peptide chain X in the compounds of the present disclosure. Hematopoietic stem cell irritants promote the proliferation of hematopoietic stem cells and supplement the reduction of blood cells such as white blood cells, red blood cells, and platelets, etc.

The term “linker” used in the present disclosure is a linking fragment for linking a polypeptide fragment X and a polypeptide fragment Y, as long as it does not affect the physiological activities of peptide chain X and peptide chain Y. There is not any limitation on its length and structure. The linker can provide a certain space for two peptide segments, so that the peptide segments tend to correctly fold without interfering with each other. The linker also provides more possibilities for interaction between the two peptide segments and promotes synergy between them. The linker includes a hydrophobic linker, a flexible hydrophilic linker, and a peptide fragment linker. The hydrophobic linker in the present disclosure is mainly amino-substituted C₁₋₈ alkyl acids, such as 4-aminobutyric acid or 6-aminohexanoic acid; the hydrophilic linker is usually a PEG polymer chain, such as (PEG)_{q}, wherein q is 1, 2, 3, 4 or 5; and the peptide fragment linker is a peptide fragment composed of 1 to 10 amino acids. From the perspective of ease of preparation, the linker is a polypeptide fragment containing 1 to 10 amino acids in length, which contains enzyme digestion sites. In some embodiments, the linker is a fragment of 2-8 amino acids in length; in some other embodiments, the linker is a fragment of 2-7 amino acids in length. In an embodiment of the present disclosure, the amino acid constituting the linker is selected from the group consisting of proline, arginine, phenylalanine, threonine, glutamic acid, asparagine, lysine, glutamine, asparagine and glycine. In a practical example of the present disclosure, the linker m is (1) (Gly-Ser)ₚ, where p is 1, 2, 3, 4 or 5; (2) (Gly-Gly-Gly-Gly-Ser)ₜ, wherein t is 1, 2 or 3; or (3) Ala-Glu-Ala-Ala-Ala-Lys-Ala. More specifically, the linker is Gly-L-Ser-Gly, (Gly-L-Ser)₂, (Gly-L-Ser)₃, or L-Ser-Gly-Gly-L-Ser-Gly-Gly-L-Ser. The linker separates the two parts of the peptide chain to reduce the steric hindrance effect between each other, and the linker can be hydrolyzed in the living body, which is beneficial to the respective active effects of peptide segment.

As used herein, the terms “chemical modification” or “capping” are used interchangeably, and indicates for the introduction of a protective group to one or both ends of a compound via a covalent modification. Suitable protecting groups serve to cap the peptide ends without reducing the biological activity of the peptide. Chemical modification can be at the amino or carboxy terminals of the compound or any residue of both, including thiol-containing amino acids.

Peptide therapeutic agents are susceptible to attack by peptidases. Exopeptidases are generally non-specific enzymes that cleave amino acid residues from the amino or carboxy terminals of a peptide or protein. Endopeptidases that cleave within amino acid sequences can also be non-specific; however, endopeptidases generally recognize specific amino sequences (recognition sites) and cleave peptides at or near those sites. Therefore, modifications to the compounds are considered to protect them from proteolytic degradation. One method of protecting a peptide from proteolytic degradation involves chemical modification or “capping” of the amino and/or carboxyl terminals of the peptide.

In some embodiments, the N-terminals and C-terminals of the peptide of the present invention may be free. When the C-terminals are free, no substituent is added or represented by “—OH”. When the C-terminals are free, no substituent is added or represented by “H”. In other embodiments, the peptide segments of the present disclosure can be chemically modified.

In some more specific embodiments, the N-acetyl peptide (which is expressed as “Ac-” in the structure or formula of the present disclosure) is produced by acetylation of chemical modification of the amino terminals of a compound. In other embodiments, a primary carboxamide (which is represented as “—NH₂” in the peptide sequence, structure or formula of the present disclosure) can be produced at the C-terminal by subjecting the carboxyl terminal of the peptide to amidation. In some embodiments, the amino- and carboxy-terminals are chemically modified by acetylation and amidation, respectively. However, other capping groups are possible. For example, the amino terminals can be capped by acylation with a group such as acetyl, benzoyl, or the like, or capped by using natural or unnatural amino acids such as β-alanine capped with acetyl, or capped by alkylation with groups such as benzyl or butyl, or capped by sulfonylation to form a sulfonamide. Similarly, the carboxy terminals can be esterified or converted into secondary amides and acylsulfonamides and the like. In some embodiments, the amino- or carboxy-terminals may comprise a site for linking a polyethylene glycol (PEG) moiety, i.e., the amino or carboxy-terminals may be chemically modified by reaction with a suitable functionalized PEG.

As used herein, “treatment” may include prophylactic and therapeutic treatments. For example, therapeutic treatment may include delaying, inhibiting or preventing the development of osteoporosis, reducing or eliminating the symptoms associated with osteoporosis. Preventive treatment may include preventing, suppressing or delaying the occurrence of osteoporosis.

A “therapeutically effective amount” as used herein refers to a dosage sufficient to cause desired response. In the present disclosure, the expected biological response is a reduction in the rate of bone loss and/or an increase in bone mass and bone mineral density in the subject.

The osteoporosis according to the present disclosure is a group of bone diseases caused by multiple causes. It is a metabolic bone disease characterized by normal calcification of bone tissue, having a normal ratio of calcium salts and matrix, and a reduction in bone tissue volume per unit volume. According to the different causes, osteoporosis can be divided into idiopathic (primary) osteoporosis and secondary osteoporosis. Therein, primary osteoporosis includes juvenile adult osteoporosis, menopausal osteoporosis and senile osteoporosis. The causes of secondary osteoporosis include: \textcircled{1} endocrine cortisol increase, hyperthyroidism, primary hyperparathyroidism, acromegaly, hypogonadism, diabetes, etc.; \textcircled{2} gestation, breastfeeding; \textcircled{3} nutritional protein deficiency, vitamin C and D deficiency, low-calcium diet, alcoholism, etc.; \textcircled{4} inherited osteogenesis imperfect chromosomal abnormalities; \textcircled{5} liver disease; \textcircled{6} kidney disease, chronic nephritis, hemodialysis; \textcircled{7} drug corticosteroids, antiepileptic drugs, antitumor drugs (such as methotrexate), heparin, etc.; \textcircled{8} decadent systemic osteoporosis, often found in a subject after long-term bed rest, paraplegia, space flight, etc., and locally found after fracture, Sudecks atrophy, bone atrophy after injury, etc.; \textcircled{9} gastrointestinal malabsorption gastrectomy; \textcircled{10} rheumatoid arthritis.

The dosage of the active polypeptide compound in the present disclosure for treating the above-mentioned diseases or disorders varies depending on the mode of administration, the age and body weight of the subject, and the health condition of the subject to be treated, and is ultimately determined by a care physician or the veterinarian. It is also considered within the scope of the present disclosure to use a peptide comprised by the above general formula for the treatment of diseases or disorders related to bone growth defects and the like, such as osteoporosis.

The active polypeptide compounds disclosed herein are generally formulated into a dosage form suitable for administration to a patient by a desired route. A therapeutically effective amount of a peptide of the present disclosure and a pharmaceutically acceptable carrier (such as magnesium carbonate, lactose, or phospholipids that cause the therapeutic compound to form a colloidal molecule) are combined to form a therapeutic composition (such as pills, tablets, capsules or liquid) to be administered (by oral, intravenous, transdermal, intrapulmonary, intravaginal, subcutaneous, intranasal, iontophoresis or transtracheal) to a subject. Pills, tablets or capsules for oral administration may be coated with a protective substance that protects the active composition from the gastric acid or enteric enzymes in the stomach for a period of time sufficient to prevent the active composition from digesting and to enter the small intestine. Therapeutic compositions may also be in the form of biodegradable or non-biodegradable sustained-release preparations for subcutaneous or intramuscular administration. See, for example, U.S. Pat. Nos. 3,773,919, 4,767,628 and PCT application WO 94/15587. Continuous administration can also be achieved by implantable or external pumps (such as INFUSADO™ pump). The administration may be performed periodically, such as once a day, or continuously at a low dosage, such as a sustained release formulation. The route of administration of the pharmaceutical composition according to the present disclosure includes, but not limited to: subcutaneous injection, subcutaneous long-acting preparation, intravenous injection, intravenous or subcutaneous infusion, intraocular injection, intradermal injection, intramuscular injection, intraperitoneal injection, intratracheal administration, intralipid administration, intra-arterial administration, intrathecal administration, epidural administration, inhalation, intranasal administration, sublingual administration, buccal administration, rectal administration, vaginal administration, intracranial and topical administration, transdermal administration or local delivery (such as via a catheter or stent). Transdermal delivery of a drug to the body is a desirable and convenient method for the systemic delivery of a biologically active substance to a subject, especially for the delivery of a substance with poor oral bioavailability (such as protein and peptide). Compounds can penetrate the outer stratum corneum of the skin via a transdermal delivery route, which acts as an effective barrier for substances to enter the body. Below the stratum corneum is a vibrant epidermis that does not contain blood vessels but has some nerves. Deeper is the dermis, which contains blood vessels, the lymphatic system, and nerves. Drugs that cross the stratum corneum barrier generally can diffuse into the capillaries of the dermis for absorption and systemic distribution.

The term “intradermal” means that in the treatment methods described herein, a therapeutically effective amount of an active polypeptide compound is applied to the skin to deliver the compound to the skin layer below the stratum corneum, thereby achieving the desired therapeutic effect. The term “subcutaneous” means that in the treatment methods described herein, a therapeutically effective amount of an active polypeptide compound is applied to the skin to deliver the compound to the subcutaneous tissue below the stratum corneum, thereby achieving the desired therapeutic effect.

The active polypeptide compounds described herein can be administered as separate active agents or can be administered in combination with other therapeutic agents, including other compounds having the same or similar therapeutic activity and determined to be safe and effective for such combination administration. In one aspect, the present disclosure provides a method of treating, preventing or improving a disease or disorder, comprising administering a safe and effective amount of a combination drug comprising an active polypeptide compound disclosed herein and one or more therapeutically active agents. In some embodiments, the combination drug comprises one or two other therapeutic agents. The other therapeutic agent is selected from the group consisting of a drug that inhibits bone resorption, a drug that promotes ossification, a drug that promotes bone mineralization, or parathyroid hormone-related proteins.

The specific combination of the therapies (treatments or procedures) used in the combination regime should take into account the compatibility of the desired treatment and/or procedure and the desired therapeutic effect to be achieved. The combination therapy as defined herein can be achieved by administering the individual components of the therapy simultaneously, sequentially or separately.

Each peptide of the present disclosure is capable of stimulating bone growth in a subject (in other words, a mammal such as a patient). Therefore, when administered alone or in combination with a drug that inhibits bone resorption, a drug that promotes bone formation, a drug that promotes bone mineralization, or parathyroid hormone-related proteins, the peptide is effective in treating osteoporosis and fractures. When the active polypeptide compound according to the present disclosure is used together with these therapeutic agents having similar effects, sequential application is more advantageous for improving bone mineral density.

### BRIEF DESCRIPTION OF DRAWINGS

In order to explain the technical solutions in the embodiments of the present disclosure or the prior art more clearly, the drawings used in the description of the examples or the prior art will be briefly introduced hereinafter.

FIG. 1 shows a scanned figure by micron X-ray 3D imaging system of trochlea of thigh bone in the Control group, Model group, abaloptide group (Aba group), 20 μg/kg dosage group of Example 1, 20 μg/kg dosage group of Example 2 and 20 μg/kg dosage group of Example 5.

### DETAILED DESCRIPTION

The present disclosure will be described in further detail below with reference to specific examples, but the embodiments of the present disclosure are not limited thereto. The embodiments of the present disclosure are given merely for the purpose of illustrating the present disclosure, rather than limiting the present disclosure. Therefore, any improvement to the present disclosure under the premise of the method of the present disclosure belongs to the protection scope of the present disclosure. Generally, the compounds of the present disclosure can be prepared by the methods described in the present disclosure. One of ordinary skill in the art can also use well-known methods to select sequential or different synthetic steps to produce polypeptide compounds having the structure described in the present disclosure. The following reaction schemes and examples are provided to further illustrate the content of the present disclosure.

One of ordinary skill in the art will recognize that the polypeptide compounds described in the present disclosure can be prepared by solid-phase synthesis (SPPS), liquid-phase synthesis, and enzymatic synthesis. The polypeptide compounds of the present disclosure prepared by different preparation methods all fall within the scope of the present disclosure. For example, peptide compounds are usually prepared by solid-phase synthesis. The solid-phase synthesis may be selected from conventional polystyrene-divinylbenzene crosslinked resins, polyacrylamides, polyethylene-glycol resins, and the like, for example: Wang Resin, Fmoc-Pro-CTC, Rink Amide Linker MBHA resin, etc. According to different linking sequences, appropriate resins are selected. For example, the carboxyl group of the carboxyl-terminal amino acid can be first covalently bonded to the polymer solid phase carrier. The protective group of the α-amino group can also be Fmoc, Boc, or Z. From C-terminal to N-terminal, the amino acids are subjected to repetitive process of de-protection, condensation, re-de-protection and condensation according to a certain sequences, giving a peptide chain resin having protective groups, which is subjected to steps of resin removal and de-protection, giving the required peptide chain. The amino of the amino acid on the amino-terminal can also be covalently bonded to the polymer solid phase carrier. By reverse synthesis, from the N-terminal to the C-terminal, the amino acids are subjected to repetitive process of de-protection, condensation, re-de-protection and condensation according to a certain sequences, giving a peptide chain resin having protective groups, which is subjected to steps of resin removal and de-protection, giving the required peptide chain. Terminals of peptide chains obtained by using different kinds of resin sometimes may differ. For example, peptide segment prepared by Wang resin have free carboxyl terminals. Similarly, peptide segment with NH₂ modified-carboxyl terminals are obtained when Rink-AM amino resin is used as a solid phase.

The amino acid raw materials required for peptide compound synthesis were purchased from GL Biochemical (Shanghai) Co., Ltd.; the solid-phase synthetic resin was purchased from Xi'an Sunresin Technology New Material Co., Ltd.; the amino acid condensation catalysts TBTU and DIEA used were purchased from Suzhou Highfine Biotechnology Co., Ltd. The eluents used in the preparation of HPLC was of chromatographic grade. The reagents were purchased from commercial suppliers such as Aldrich Chemical Company, Arco Chemical Company and Alfa Chemical Company, and were used without further purification, unless otherwise indicated. The analysis and detection instruments used are conventional instruments and equipment in the field. In the examples described below, unless otherwise indicated that all temperatures are set to degrees Celsius, and the given temperature may have a fluctuation range of ±5° C.

When identifying the structure of the peptide compound, QE identification, N-terminal analysis of protein (by mass spectrometry) and N-terminal sequence analysis of polypeptide protein were used to confirm the primary structure, and circular dichroism scanning analysis was used to confirm the secondary structure.

In the examples of the present disclosure, the circular dichroism scanning analysis of polypeptide compound is performed with a Chirascan Plus V100 circular dichroism spectrometer (British Applied Optics) to collect the circular dichroism (CD) absorption spectra of the protein test product in far ultraviolet (190-260 nm) and near ultraviolet (250-340 nm) regions, and to analyze the secondary structure by software. In the specific measurement, a scanning wavelength of 180-340 nm was set for background test and blank buffer test, and then circular dichroism far and near ultraviolet absorptions of a 1 mg/mL CSA standard solution was collected in the range of 180-340 nm. All scanned spectra were subjected to subtract baseline and smoothing treatments with software Pro-Data Viewer. The ratio of the peak and valley CD values of a standard sample was calculated, and the effective ratio range is 2.08±0.06. CDNN software was used to fit the secondary structure of the test sample, and the proportions of helix, antiparallel+parallel, beta-turn, and random coil in different wavelength intervals were calculated in the Milli-Degress mode.

In the QE identification of the polypeptide compound in examples of the present disclosure, protein polypeptide was subjected to enzymolysis with an endonuclease (generally Trypsin), and then LC/MS/MS (nanoLC-QE) was used to analyze the sample after enzymolysis. Finally, the mass spectrometry software such as MASCOT was used to analyze the LC/MS/MS data to obtain the qualitative identification information of the target protein and peptide molecules. In the specific measurement, after the test product was reduced and alkylated, Trypsin (in a mass ratio of 1:50) was added, and enzymolysis was carried out at 37° C. for 20 hours. The enzymolysis product was desalted, freeze-dried, re-dissolved in a 0.1% FA solution, and stored at −20° C. until use. Q Exactive (Thermo Fisher) and Easy-nLC 1000 (Thermo Fisher) were used. The mass-to-charge ratios of polypeptides and polypeptide fragments were collected as follows: 20 fragment spectra (MS2 scan) were collected after each full scan. The raw file of mass spectrometry test was used to search the corresponding database with Mascot2.2 software, finally giving the identified protein results.

In the examples of the present disclosure, the experimental method of protein N-terminal sequence analysis (by mass spectrometry) of polypeptide compound was performed by: subjecting the protein to enzymolysis respectively with trypsin, chymotrypsin and Glu-C enzyme, and then using LC-MS/MS (Xevo G2-XS QTof, Waters) to analysis the peptide segment sample after enzymolysis. Enzymolysis method: 50 μg of test product was dissolved in in an appropriate amount of guanidine hydrochloride to denature, then after DTT and IAM reactions, the disulfide bond was reduced and protected by alkylation modification, and 1 μg of trypsin, 1 μg of chymotrypsin and 1 μg of Glu-C enzyme were added after dilution, reacted at 37° C. for 20 hours. Finally, the UNIFI software was used to analyze the LC-MS/MS data, and the N-terminal amino acid sequence of the test product was determined to whether be in accordance with the theoretical sequence based on the results of the algorithm. For the specific measurement, the instruments were (1) high-resolution mass spectrometer: XevoG2-XS QTof (Waters), and (2) ultra-high performance liquid chromatography: UPLC (Acquity UPLC I-Class) (Waters).

The N-terminal sequence analysis of the polypeptide protein of the polypeptide compound in the examples of the present disclosure was performed by analyzing the N-terminal sequence of the test product by a fully automatic protein peptide sequencer. The PPSQ fully automatic protein peptide sequencer (SHIMADZU) was used in the examples of the present disclosure. Sample name, sample number, number of test cycles and selection of a method file were set by software PPSQ Analysis, and the test started after the settings were completed. Data and Atlas Processing: the raw data and spectra generated by PPSQ were identified, peaks were marked up by PPSQ Data Processing software, and the corresponding spectra were derived.

In the examples of the present disclosure, the preliminary structure of the polypeptide compound was determined by mass spectrometry. High-resolution mass spectrometry was performed with ABSciex 5800 MALDI-TOF/TOF to test the relative molecular mass of the protein, and accurate and reliable relative molecular mass information of the polypeptide was obtained.

The following abbreviations are used throughout the disclosure:

Boc: tert-butoxycarbonyl

DIEA: diisopropylethylamine

DCM: dichloromethane

CH₃CN: acetonitrile

DCM: dichloromethane

DMF: N,N-dimethylformamide

DEPBT: 3-(diethoxy orthoacyloxy)-1,2,3-benzotriazin-4-one

DIEA: diisopropylethylamine

Et₂O: ethyl ether

EDT: Ethylene Dithiol

Fmoc: 9H-fluoren-9-ylmethoxycarbonyl

H₂O: water

HBTU: 2-(1H-benzotriazol-1-yl-)-1,1,3,3-tetramethylurea hexafluorophosphate

NMP: 1-methyl-pyrrolidin-2-one

Ot-Bu: tert-butoxy

PyBOP: 1H-benzotriazol-1-yloxytripyrrolidinyl hexafluorophosphate

Pbf: 2,2,4,6,7-pentamethylbenzodihydrofuran-5-sulfonyl

t-Bu: tert-butyl

Trt: Trityl

TIS: Triisopropyl silane

T₃P: 1-propyl phosphoric anhydride

TFA: trifluoroacetic acid

Trt: trityl

rt: room temperature

TA: thioanisole

### Examples of Preparation

In the following specific examples, the peptides in the present disclosure can be prepared by standard solid-phase synthesis method. The preparing process will be described in detail hereinafter. Other peptides in the present disclosure can be prepared by a similar method by one of ordinary skill in the art.

Example 1:
(the polypeptide sequence shown as SEQ ID No.9)
Preparation of Ala-Val-Ser-Glu-His-Gln-Leu-Leu-
His-Asp-Lys-Gly-Lys-Ser-Ile-Gln-Asp-Leu-Arg-Arg-
Arg-Glu-Leu-Leu-Glu-Lys-Leu-Leu-(N-Me)Ala-Lys-
Leu-His-Thr-Ala-Tyr-Gly-Phe-Gly-Gly

This example was synthesized from C terminal to N terminal.

(1-1) Preprocessing of resin: 30 g 2-CTA resin (degree of substitution 0.93 mmol/g, 27 mmol) was weighed, and placed in a 250 mL test tube. The resin was swelled with 180 mL DCM for 30 min. The solvent was pumped off in vacuum. Then DCM (100 mL) was added in, nitrogen was introduced in and heated, until the temperature reached 25° C. SOCl₂ (10 mL, 5.0 eq) was added dropwise, the temperature was kept at 30-35° C., and reacted for 2 h. After the reaction was completed, nitrogen was introduced in to press and solvent was pumped off in vacuum. DCM (100 mL×3) was added to wash the resin, and the solvent was pumped off each time after washing.

(1-2) Linking S1 amino acid: Fmoc-Gly-OH (32.5 g, 108 mmol) was weighed, and dissolved in 100 mL DCM. After dissolved completely, DIEA (23 mL, 135 mmol) was added. The obtained mixture was placed in a test tube, nitrogen was introduced, the mixture was stirred and reacted at 20° C.-30° C. for 2 h. 12 mL of mixed solvent of methanol and DIEA (methanol:DIEA=9:1) was added droppedwise, to seal the unreacted sites for 10 min. The solvent was pumped off. The resin was washed with DCM (150 mL×2). After washing, the solvent was pumped off. Then DMF (150 mL×2) was used to wash the resin, and the solvent was pumped off after washing. The resin was then swelled with 120 mL DMF for 30 min. The solvent was pumped off, and de-protection was performed with a piperidine/DMF solution (120 mL) having a volume ratio of 20% for twice. The times were respectively 10 min and 15 min. The temperature of the reaction was controlled at 20° C.-30° C. After the de-protection, the solvent was pumped off, and the resin was washed with DMF (120 mL×6). After washing, the solvent was pumped off, and the resin was left in the test tube. Ninhydrin was used to test the color of resin, and the resin was purple-black. The next step was carried out. The absorbance was detected by spectrophotometric method, and the degree of substitution of the resin was calculated to be 0.8641 mmol/g.

(1-3) Linking S2 Amino Acid:

Fmoc-Gly-OH (19.0 g, 63.9 mmol, 3.0 eq.) and PyBOP (33.25 g, 63.9 mmol, 3.0 eq.) were weighted, and dissolved with 50 mL DMF. After dissolved completely, DIEA (10.5 mL, 63.9 mmol) was added. The obtained mixture was placed in a test tube, nitrogen was introduced, the mixture was stirred and reacted at 20° C.-30° C. for 2 h. Ninhydrin was used to test the color of resin, the resin was transparent yellow. After the reaction, the solvent was pumped off, and the resin was washed with DMF (120 mL×3). After washing, the solvent was pumped off, and then de-protection was performed with a piperidine/DMF solution (120 mL) having a volume ratio of 20% for twice. The times were respectively 10 min and 15 min. The temperature of the reaction was controlled at 20° C.-30° C. After the de-protection, the solvent was pumped off, and the resin was washed with DMF (120 mL×6). After washing, the solvent was pumped off, and the resin was left in the test tube. Ninhydrin was used to test the color of resin, and the resin was purple-black. The next step was carried out.

(1-4) Linking S3 Amino Acid:

Fmoc-Phe-OH (24.75 g, 63.9 mmol, 3.0 eq.) and PyBOP (33.25 g, 63.9 mmol, 3.0 eq.) were weighted, and dissolved in 50 mL DMF. After dissolved completely, DIEA (10.5 mL, 63.9 mmol) was added. The obtained mixture was placed in a test tube, nitrogen was introduced, the mixture was stirred and the temperature was controlled at 20° C.-30° C. and reacted for 2 h. Ninhydrin was used to test the color of resin, the resin was transparent yellow. After the reaction, the solvent was pumped off, and the resin was washed with DMF (120 mL×3). After washing, the solvent was pumped off, and then de-protection was performed with a piperidine/DMF solution (120 mL) having a volume ratio of 20% for twice. The times were respectively 10 min and 15 min, and the temperature of the reaction was controlled at 20° C.-30° C. After the de-protection, the solvent was pumped off, and the resin was washed with DMF (120 mL×6). After washing, the solvent was pumped off, and the resin was left in the test tube. Ninhydrin was used to test the color of resin, and the resin was purple-black. The next step was carried out.

(1-5) Steps (1-4) were repeated. S4 amino acid Fmoc-Gly-OH, S5 amino acid Fmoc-Tyr(t-Bu)-OH, S6 amino acid Fmoc-Ala-OH, S7 amino acid Fmoc-Thr(t-Bu)-OH, S8 amino acid Fmoc-His(trt)-OH, S9 amino acid Fmoc-Leu-OH, S10 amino acid Fmoc-Lys(Boc)-OH, S11 amino acid Fmoc-(N-Me)Ala-OH, S12 amino acid Fmoc-Leu-OH, S13 amino acid Fmoc-Leu-OH, S14 amino acid Fmoc-Lys(Boc)-OH, S15 amino acid Fmoc-Glu(Ot-Bu)-OH, S16 amino acid Fmoc-Leu-OH, S17 amino acid Fmoc-Leu-OH, S18 amino acid Fmoc-Glu(Ot-Bu)-OH, S19 amino acid Fmoc-Arg(pbf)-OH, S20 amino acid Fmoc-Arg(pbf)-OH, S21 amino acid Fmoc-Arg(pbf)-OH, S22 amino acid Fmoc-Leu-OH, S23 amino acid Fmoc-Asp(OtBu)-OH, S24 amino acid Fmoc-Gln(trt)-OH, S25 amino acid Fmoc-Ile-OH, S26 amino acid Fmoc-Ser(tBu)-OH, S27 amino acid Fmoc-Lys(Boc)-OH, S28 amino acid Fmoc-Gly-OH, S29 amino acid Fmoc-Lys(Boc)-OH, S30 amino acid Fmoc-Asp(Ot-Bu)-OH, S31 amino acid Fmoc-His(trt)-OH, S32 amino acid Fmoc-Leu-OH, S33 amino acid Fmoc-Leu-OH, S34 amino acid Fmoc-Gln(trt)-OH, S35 amino acid Fmoc-His(trt)-OH, S36 amino acid Fmoc-Glu(Ot-Bu)-OH, S37 amino acid Fmoc-Ser(t-Bu)-OH, S38 amino acid Fmoc-Val-OH and S39 amino acid Fmoc-Ala-OH were successively linked, and a peptide resin was obtained to carry out the next operation.

(1-6) Resin shrinkage: methanol (80 mL) was firstly added in the test tube, the resin was shrunken for 5 min, and the solvent was pumped off. The shrinkage was repeated for 3 times, 10 min once. Each time after the shrinkage, the solvent was pumped out completely before the next shrinkage. Then the shrunken resin was placed in a vacuum drying oven, dried at 35° C., and 18.82 g peptide resin was obtained.

(1-7) Peptide segment cracking: 155 mL TFA, 8 mL TIS, 4.12 mL EDT, 2 mL TA, 4.12 mL water and 2 mL anisole were mixed evenly to prepare the lysate. 18.82 g of the peptide resin prepared in step (1-6) was weighed, the lysate and the peptide resin were mixed, sealed and shielded from the light. The mixture was stirred and reacted, the temperature was kept at 25° C.-35° C., and reacted for 2 h. After the reaction, a sand core funnel was used to remove the resin. After removing the solvent in vacuum, methyl tertiary butyl ether (450 mL) was added in the rest liquid, and crystallized at 0° C.-10° C. for 2 h. The mixture was centrifuged to remove the crystallizing solution, and the precipitate was washed with methyl tertiary butyl ether for 3 times. The precipitate was collected, and dried in vacuum at 35° C., to give a polypeptide (SEQ ID NO:9) Ala-Val-Ser-Glu-His-Gln-Leu-Leu-His-Asp-Lys-Gly-Lys-Ser-Ile-Gln-Asp-Leu-Arg-Arg-Arg-Glu-Leu-Leu-Glu-Lys-Leu-Leu-(N-Me)Ala-Lys-Leu-His-Thr-Ala-Tyr-Gly-Phe-Gly-Gly.

(1-8) Purification: after filtering the peptide crude solution obtained in step (1-7) with a 0.45 μm filter membrane, the solution was subjected to preparative HPLC purification in a 20 mm×150 mm column filled with 10 μm C-18 silica gel. The detection wavelength was 220 nm. The mobile phase A was 0.1% TFA, and the mobile phase B was acetonitrile. Gradient elution was carried out according to the following Table A.

TABLE A
Gradient elution program

| | Flow rate | Mobile | Mobile |
|---|---|---|---|
| Time | (mL/ | phase | phase |
| (min) | min) | A (%) | B (%) |
| 0 | 8 | 95 | 5 |
| 0.1 | 8 | 75 | 25 |
| 45 | 8 | 65 | 50 |
| 60 | 8 | 50 | 50 |

Fractions containing target polypeptide product was collected, and the purity was 95.8%. The collected fractions were combined, the solvent was removed in vacuum, and the polypeptide compound was freeze-dried. The obtained end product was identified by analytical RP-HPLC (retention time), LC-MS and MALDI/TOF-MS.

MALDI/TOF-MS(ESI): 4441.2236 [M+H]⁺.

According to QE identification and analysis, the sequence of the obtained polypeptide compound was as that shown as SEQ ID NO:9.

Assay: moisture content was measured with a moisture titrator by the moisture determination method in Chinese Pharmacopoeia, and TFA content was measured by the acetic acid content detection method in Chinese Pharmacopoeia, and the content of the polypeptide was 83.7%.

Example 2:
(the polypeptide sequence shown as SEQ ID No. 10)
Preparation of Ala-Val-Ser-Glu-His-Gln-Leu-Leu-
His-Asp-Lys-Gly-Lys-Ser-Ile-Gln-Asp-Leu-Arg-Arg-
Arg-Glu-Leu-Leu-Glu-Lys-Leu-Leu-Aib-Lys-Leu-His-
Thr-Ala-Tyr-Gly-Phe-Gly-Gly

The compound was synthesized from C terminal to N terminal:

(2-1) Preprocessing of resin: 30 g 2-CTA resin (degree of substitution 0.93 mmol/g, 27 mmol) was weighed, disposed in a 250 mL test tube, and swelled in DCM (200 mL) for 30 min. The solvent was pumped out in vacuum. Then DCM (100 mL) was added, nitrogen was introduced, and heated until the temperature reached 25° C. SOCl₂ (10 mL, 5.0 eq) was added dropwise, the temperature was kept at 30-35° C., and the reaction was performed for 2 h. After the reaction was complete, nitrogen was introduced in and solvent was pumped off. DCM (100 mL×3) was added to wash the resin, and the solvent was pumped off each time after washing.

(2-2) Linking S1 amino acid: Fmoc-Gly-OH (32.5 g, 108 mmol) was weighed and dissolved in 100 mL DCM. After dissolved completely, DIEA (23 mL, 135 mmol) was added. The obtained mixture was added in a test tube, nitrogen was introduced, stirred and reacted at 20° C.-30° C. for 2 h. 12 mL of mixed solvent of methanol and DIEA (methanol:DIEA=9:1) was added, and the unreacted sites was sealed for 10 min. The solvent was pumped off. The resin was washed with DCM (150 mL×2). After washing, the solvent was pumped off. Then DMF (150 mL×2) was used to wash the resin, and the solvent was pumped off after washing. The resin was then swelled with 120 mL DMF for 30 min. The solvent was pumped off, and de-protection was performed with a piperidine/DMF solution (120 mL) having a volume ratio of 20% for twice. The times were respectively 10 min and 15 min, and the temperature of the reaction was controlled at 20° C.-30° C. After the de-protection, the solvent was pumped off, and the resin was washed with DMF (120 mL×6). After washing, the solvent was pumped off, and the resin was left in the test tube. Ninhydrin was used to test the color of resin, and the resin was purple-black. The next step was carried out. The absorbance was detected by spectrophotometric method, and degree of substitution of the resin was calculated to be 0.8641 mmol/g.

(2-3) Linking S2 Amino Acid:

Fmoc-Gly-OH (19.0 g, 63.9 mmol, 3.0 eq.) and DEPBT (19.17 g, 63.9 mmol, 3.0 eq.) were weighed, and dissolved in 50 mL DMF. After dissolved completely, DIEA (10.5 mL, 63.9 mmol) was added. The obtained mixture was added in a test tube, nitrogen was introduced, stirred and reacted at 20° C.-30° C. for 2 h. Ninhydrin was used to test the color of resin, the resin was transparent yellow. After the reaction was completed, the solvent was pumped off, and the resin was washed with DMF (120 mL×3). The solvent was pumped off after washing, and de-protection was performed with a piperidine/DMF solution (120 mL) having a volume ratio of 20% for twice. The times were respectively 10 min and 15 min, and the temperature of the reaction was controlled at 20° C.-30° C. After the de-protection, the solvent was pumped off, and the resin was washed with DMF (120 mL×6). After washing, the solvent was pumped off, and the resin was left in the test tube. Ninhydrin was used to test the color of resin, and the resin was purple-black. The next step was carried out.

(2-4) Linking S3 Amino Acid:

Fmoc-Phe-OH (24.75 g, 63.9 mmol, 3.0 eq.) and DEPBT (19.17 g, 63.9 mmol, 3.0 eq.) were weighed, dissolved in 50 mL DMF. After dissolved completely, DIEA (10.5 mL, 63.9 mmol) was added. The obtained mixture was added in a test tube, nitrogen was introduced, stirred and reacted at 20° C.-30° C. for 2 h. Ninhydrin was used to test the color of resin, the resin was transparent yellow. After the reaction, the solvent was pumped off, the resin was washed with DMF (120 mL×3), and the solvent was pumped off, and then de-protection was performed with a piperidine/DMF solution (120 mL) having a volume ratio of 20% for twice. The times were respectively 10 min and 15 min, and the temperature of the reaction was controlled at 20° C.-30° C. After the de-protection, the solvent was pumped off, and the resin was washed with DMF (120 mL×6). After washing, the solvent was pumped off, and the resin was left in the test tube. Ninhydrin was used to test the color of resin, and the resin was purple-black. The next step was carried out.

(2-5) Steps (2-4) were repeated. S4 amino acid Fmoc-Gly-OH, S5 amino acid Fmoc-Tyr(t-Bu)-OH, S6 amino acid Fmoc-Ala-OH, S7 amino acid Fmoc-Thr(t-Bu)-OH, S8 amino acid Fmoc-His(trt)-OH, S9 amino acid Fmoc-Leu-OH, S10 amino acid Fmoc-Lys(Boc)-OH, S11 amino acid Fmoc-Aib-OH, S12 amino acid Fmoc-Leu-OH, S13 amino acid Fmoc-Leu-OH, S14 amino acid Fmoc-Lys(Boc)-OH, S15 amino acid Fmoc-Glu(Ot-Bu)-OH, S16 amino acid Fmoc-Leu-OH, S17 amino acid Fmoc-Leu-OH, S18 amino acid Fmoc-Glu(Ot-Bu)-OH, S19 amino acid Fmoc-Arg(pbf)-OH, S20 amino acid Fmoc-Arg(pbf)-OH, S21 amino acid Fmoc-Arg(pbf)-OH, S22 amino acid Fmoc-Leu-OH, S23 amino acid Fmoc-Asp(Ot-Bu)-OH, S24 amino acid Fmoc-Gln(trt)-OH, S25 amino acid Fmoc-Ile-OH, S26 amino acid Fmoc-Ser(t-Bu)-OH, S27 amino acid Fmoc-Lys(Boc)-OH, S28 amino acid Fmoc-Gly-OH, S29 amino acid Fmoc-Lys(Boc)-OH, S30 amino acid Fmoc-Asp(Ot-Bu)-OH, S31 amino acid Fmoc-His(trt)-OH, S32 amino acid Fmoc-Leu-OH, S33 amino acid Fmoc-Leu-OH, S34 amino acid Fmoc-Gln(trt)-OH, S35 amino acid Fmoc-His(trt)-OH, S36 amino acid Fmoc-Glu(Ot-Bu)-OH, S37 amino acid Fmoc-Ser(t-Bu)-OH, S38 amino acid Fmoc-Val-OH and S39 amino acid Fmoc-Ala-OH were successively linked, and a peptide resin was obtained to carry out the next operation.

(2-6) Resin shrinkage: methanol (80 mL) was firstly added in the test tube, the resin was shrunken for 5 min, and the solvent was pumped off. The shrinkage was repeated for 3 times, 10 min once. Each time after the shrinkage, the solvent was pumped out completely before the next shrinkage. Then the shrunken resin was placed in a vacuum drying oven, dried at 35° C., and 18.82 g peptide resin was obtained.

(2-7) Peptide segment cracking: 155 mL TFA, 8 mL TIS, 4.12 mL EDT, 2 mL TA, 4.12 mL water and 2 mL anisole were mixed evenly to prepare the lysate. 18.82 g of the peptide resin prepared in steps (2-6) was weighed, the lysate and the peptide resin were mixed, sealed and shielded from the light. The mixture was stirred and reacted, the temperature was kept at 25° C.-35° C., and reacted for 2 h. After the reaction, a sand core funnel was used to remove the resin. After removing the solvent in vacuum, methyl tertiary butyl ether (450 mL) was added in the rest liquid, and crystallization was performed at low temperature (0° C.-10° C.) for 2 h. The resultant mixture was centrifuged to remove the crystallizing solution, and the precipitate was washed with methyl tertiary butyl ether for 3 times. The precipitate was collected, and dried in vacuum at 35° C., and a polypeptide (SEQ ID NO:10) Ala-Val-Ser-Glu-His-Gln-Leu-Leu-His-Asp-Lys-Gly-Lys-Ser-Ile-Gln-Asp-Leu-Arg-Arg-Arg-Glu-Leu-Leu-Glu-Lys-Leu-Leu-Aib-Lys-Leu-His-Thr-Ala-Tyr-Gly-Phe-Gly-Gly was obtained.

(2-8) Purification: after filtering the peptide crude solution obtained in steps (2-7) with a 0.45 μm filter membrane, the solution was subjected to preparative HPLC purification in a 20 mm×150 mm column filled with 10 μm C-18 silica gel. The detection wavelength was 220 nm. The mobile phase A was 0.1% TFA, and the mobile phase B was acetonitrile. Gradient elution was carried out according to the following Table A.

TABLE A
Gradient elution program

| | Flow rate | Mobile | Mobile |
|---|---|---|---|
| Time | (mL/ | phase | phase |
| (min) | min) | A (%) | B (%) |
| 0 | 8 | 95 | 5 |
| 0.1 | 8 | 75 | 25 |
| 45 | 8 | 65 | 50 |
| 60 | 8 | 50 | 50 |

Fractions containing target polypeptide product were collected, and the purity was 95.8%. The collected fractions were combined, the solvent was removed in vacuum, and the polypeptide compound was freeze-dried. The obtained end product was identified by analytical RP-HPLC (retention time), LC-MS and MALDI/TOF-MS.

LC-MS(ESI): m/z 1112.2 [M/4+H]⁺.

MALDI/TOF-MS(ESI): m/z 4441.4175 [M+H]⁺.

According to QE identification and analysis, the sequence of the obtained polypeptide compound was as that shown as SEQ ID NO:10.

Assay: moisture content was measured with a moisture titrator by the moisture determination method in Chinese Pharmacopoeia, and TFA content was measured by the acetic acid content detection method in Chinese Pharmacopoeia, and the content of polypeptide was 85.9%.

Example 3:
(the polypeptide sequence shown as SEQ ID No. 11)
Preparation of Ala-Val-Ser-Glu-His-Gln-Leu-Leu-
His-Asp-Lys-Gly-Lys-Ser-Ile-Gln-Asp-Leu-Arg-Arg-
Arg-Glu-Leu-Leu-Glu-Lys-Leu-Leu-Ala-Lys-Leu-His-
Thr-Ala-Tyr-Arg-Gly-Phe-Gly-Gly

The compound was synthesized from C terminal to N terminal:

(3-1) Preprocessing of resin: 30 g 2-CTA resin was weighed (degree of substitution 0.93 mmol/g, 27 mmol), placed in a 250 mL test tube, and swelled in DCM (200 mL) for 30 min. The solvent was pumped out in vacuum. Then DCM (100 mL) was added, nitrogen was introduced, and heated until the temperature reached 25° C. SOCl₂ (10 mL, 5.0 eq) was added dropwise, the temperature was kept at 30-35° C., and reacted for 2 h. After the reaction was completed, nitrogen was introduced and the solvent was pumped off. DCM (100 mL×3) was added to wash the resin, and the solvent was pumped off each time after washing.

(3-2) Linking S1 amino acid: Fmoc-Gly-OH (32.5 g, 108 mmol) was weighed and dissolved in 100 mL DCM. After dissolved completely, DIEA (23 mL, 135 mmol) was added. The obtained mixture was added in a test tube, nitrogen was introduced, stirred and reacted at 20° C.-30° C. for 2 h. 12 mL of mixed solvent of methanol and DIEA (methanol:DIEA=9:1) was added dropwise, and the unreacted sites were sealed for 10 min. The solvent was pumped off. The resin was washed with DCM (150 mL×2). After washing, the solvent was pumped off. Then DMF (150 mL×2) was used to wash the resin, and the solvent was pumped off after washing. The resin was then swelled with 120 mL DMF for 30 min. The solvent was pumped off, and de-protection was performed with a piperidine/DMF solution (120 mL) having a volume ratio of 20% for twice. The times were respectively 10 min and 15 min, and the temperature of the reaction was controlled at 20° C.-30° C. After the de-protection, the solvent was pumped off, and the resin was washed with DMF (120 mL×6). After washing, the solvent was pumped off, and the resin was left in the test tube. Ninhydrin was used to test the color of resin, and the resin was purple-black. The next step was carried out. The absorbance was detected by spectrophotometric method, and degree of substitution of the resin was calculated to be 0.8641 mmol/g.

(3-3) Linking S2 Amino Acid:

Fmoc-Gly-OH (19.0 g, 63.9 mmol, 3.0 eq.) and T₃P (20.33 g, 63.9 mmol, 3.0 eq.) were weighed, and dissolved in 50 mL DMF. After dissolved completely, DIEA (10.5 mL, 63.9 mmol) was added. The obtained mixture was added in a test tube, nitrogen was introduced, stirred and reacted at 20° C.-30° C. for 2 h. Ninhydrin was used to test the color of resin, the resin was transparent yellow. After the reaction was completed, the solvent was pumped off, and the resin was washed with DMF (120 mL×3), 3 min each time. The solvent was pumped off after washing, and de-protection was performed with a piperidine/DMF solution (120 mL) having a volume ratio of 23% for twice. The times were respectively 10 min and 15 min, and the temperature of the reaction was controlled at 20° C.-30° C. After the de-protection, the solvent was pumped off, and the resin was washed with DMF (120 mL×6). After washing, the solvent was pumped off, and the resin was left in the test tube. Ninhydrin was used to test the color of resin, and the resin was purple-black. The next step was carried out.

(3-4) Linking S3 Amino Acid

Fmoc-Phe-OH (24.75 g, 63.9 mmol, 3.0 eq.) and T₃P (19.17 g, 63.9 mmol, 3.0 eq.) were weighed, dissolved in 50 mL DMF. After dissolved completely, DIEA (10.5 mL, 63.9 mmol) was added. The obtained mixture was added in a test tube, nitrogen was introduced, stirred and reacted at 20° C.-30° C. for 2 h. Ninhydrin was used to test the color of resin, the resin was transparent yellow. After the reaction was completed, the solvent was pumped off, the resin was washed with DMF (120 mL×3), and the solvent was pumped off after washing, and then de-protection was performed with a piperidine/DMF solution (120 mL) having a volume ratio of 20% for twice. The times were respectively 10 min and 15 min, and the temperature of the reaction was controlled at 20° C.-30° C. After the de-protection, the solvent was pumped off, and the resin was washed with DMF (120 mL×6). After washing, the solvent was pumped off, and the resin was left in the test tube. Ninhydrin was used to test the color of resin, and the resin was purple-black. The next step was carried out.

(3-5) Steps (3-4) were repeated. S4 amino acid Fmoc-Gly-OH, S5 amino acid Fmoc-Arg(pbf)-OH, S6 amino acid Fmoc-Tyr(t-Bu)-OH, S7 amino acid Fmoc-Ala-OH, S8 amino acid Fmoc-Thr(t-Bu)-OH, S9 amino acid Fmoc-His(trt)-OH, S10 amino acid Fmoc-Leu-OH, S11 amino acid Fmoc-Lys(Boc)-OH, S12 amino acid Fmoc-Ala-OH, S13 amino acid Fmoc-Leu-OH, S14 amino acid Fmoc-Leu-OH, S15 amino acid Fmoc-Lys(Boc)-OH, S16 amino acid Fmoc-Glu(Ot-Bu)-OH, S17 amino acid Fmoc-Leu-OH, S18 amino acid Fmoc-Leu-OH, S19 amino acid Fmoc-Glu(Ot-Bu)-OH, S20 amino acid Fmoc-Arg(pbf)-OH, S21 amino acid Fmoc-Arg(pbf)-OH, S22 amino acid Fmoc-Arg(pbf)-OH, S23 amino acid Fmoc-Leu-OH, S24 amino acid Fmoc-Asp(Ot-Bu)-OH, S25 amino acid Fmoc-Gln(trt)-OH, S26 amino acid Fmoc-Ile-OH, S27 amino acid Fmoc-Ser(t-Bu)-OH, S28 amino acid Fmoc-Lys(Boc)-OH, S29 amino acid Fmoc-Gly-OH, S30 amino acid Fmoc-Lys(Boc)-OH, S31 amino acid Fmoc-Asp(Ot-Bu)-OH, S32 amino acid Fmoc-His(trt)-OH, S33 amino acid Fmoc-Leu-OH, S34 amino acid Fmoc-Leu-OH, S35 amino acid Fmoc-Gln(trt)-OH, S36 amino acid Fmoc-His(trt)-OH, S37 amino acid Fmoc-Glu(Ot-Bu)-OH, S38 amino acid Fmoc-Ser(t-Bu)-OH, S39 amino acid Fmoc-Val-OH and S40 amino acid Fmoc-Ala-OH were successively connected, and a peptide resin was obtained to carry out the next operation.

(3-6) Resin shrinkage: methanol (80 mL) was firstly added in the test tube, the resin was shrunken for 5 min, and the solvent was pumped off. The shrinkage was repeated for 3 times, 10 min once. Each time after the shrinkage, the solvent was pumped out completely before the next shrinkage. Then the shrunken resin was placed in a vacuum drying oven, dried at 35° C., and 19.56 g peptide resin was obtained.

(3-7) Peptide segment cracking: 155 mL TFA, 8 mL TIS, 4.12 mL EDT, 2 mL TA, 4.12 mL water and 2 mL anisole were mixed evenly to prepare the lysate. 18.82 g of the peptide resin prepared in steps (3-6) was weighed, the lysate and the peptide resin were mixed, sealed and shielded from the light. The mixture was stirred and reacted, the temperature was kept at 25° C.-35° C., and reacted for 2 h. After the reaction was completed, a sand core funnel was used to remove the resin. After removing the solvent in vacuum, methyl tertiary butyl ether (450 mL) was added in the rest liquid, and crystallization was performed at low temperature (0° C.-10° C.) for 2 h. The mixture was centrifuged to remove the crystallizing solution, and the obtained precipitate was washed with methyl tertiary butyl ether for 3 times. The precipitate was collected, and dried in vacuum at 35° C., to obtain polypeptide (SEQ ID NO:11) Ala-Val-Ser-Glu-His-Gln-Leu-Leu-His-Asp-Lys-Gly-Lys-Ser-Ile-Gln-Asp-Leu-Arg-Arg-Arg-Glu-Leu-Leu-Glu-Lys-Leu-Leu-Ala-Lys-Leu-His-Thr- Ala-Tyr-Arg-Gly-Phe-Gly-Gly.

(3-8) Purification: after filtering the peptide crude solution obtained in steps (3-7) with a 0.45 μm filter membrane, the mixture was subjected to preparative HPLC purification in a 20 mm×150 mm column filling with 10 μm C-18 silica gel. The detection wavelength was 220 nm. The mobile phase A was 0.1% TFA, and the mobile phase B was acetonitrile. Gradient elution was carried out according to the following Table A.

TABLE A
Gradient elution program

| | Flow rate | Mobile | Mobile |
|---|---|---|---|
| Time | (mL/ | phase | phase |
| (min) | min) | A(%) | B(%) |
| 0 | 8 | 95 | 5 |
| 0.1 | 8 | 75 | 25 |
| 45 | 8 | 65 | 50 |
| 60 | 8 | 50 | 50 |

Fractions containing target polypeptide product were collected, and the purity was 96.1%. The collected fractions were combined, the solvent was removed in vacuum, and the polypeptide compound was freeze-dried. The obtained end product was identified by analytical RP-HPLC (retention time) and MALDI/TOF-MS.

MALDI/TOF-MS(ESI): m/z 4585.2 [M+H]⁺.

According to QE identification and analysis, the sequence of the obtained polypeptide compound was as that shown as SEQ ID NO: 11.

Assay: moisture content was measured with a moisture titrator by the moisture determination method in Chinese Pharmacopoeia, and TFA content was measured by the acetic acid content detection method in Chinese Pharmacopoeia, and the content of polypeptide was 81.97%.

Example 4:
(the polypeptide sequence shown as SEQ ID No. 12)
Preparation of Ala-Val-Ser-Glu-His-Gln-Leu-Leu-
His-Asp-Lys-Gly-Lys-Ser-Ile-Gln-Asp-Leu-Arg-Arg-
Arg-Phe-Phe-Leu-His-His-Leu-Ile-Ala-Glu-Ile-His-
Thr-Ala-Tyr-Gly-Phe-Gly-Gly

The process as shown in Example 2 was adopted. 2-CTA resin having a degree of substitution of 0.93 mmol/g was used. The resin was firstly swelled to prepare 2-CTA resin into CTC resin. Then the side chain-protected amino acids Fmoc-Gly-OH, Fmoc-Gly-OH, Fmoc-Phe-OH, Fmoc-Gly-OH, Fmoc-Tyr(t-Bu)-OH, Fmoc-Ala-OH, Fmoc-Thr(t-Bu)-OH, Fmoc-His(trt)-OH, Fmoc-Ile-OH, Fmoc-Glu(Ot-Bu)-OH, Fmoc-Ala-OH, Fmoc-Ile-OH, Fmoc-Leu-OH, Fmoc-His(trt)-OH, Fmoc-His(trt)-OH, Fmoc-Leu-OH, Fmoc-Phe-OH, Fmoc-Phe-OH, Fmoc-Arg(pbf)-OH, Fmoc-Arg(pbf)-OH, Fmoc-Arg(pbf)-OH, Fmoc-Leu-OH, Fmoc-Asp(Ot-Bu)-OH, Fmoc-Gln(trt)-OH, Fmoc-Ile-OH, Fmoc-Ser(t-Bu)-OH, Fmoc-Lys(Boc)-OH, Fmoc-Gly-OH, Fmoc-Lys(Boc)-OH, Fmoc-Asp(Ot-Bu)-OH, Fmoc-His(trt)-OH, Fmoc-Leu-OH, Fmoc-Leu-OH, Fmoc-Gln(trt)-OH, Fmoc-His(trt)-OH, Fmoc-Glu(Ot-Bu)-OH, Fmoc-Ser(t-Bu)-OH, Fmoc-Val-OH, Fmoc-Ala-OH were successively linked to give the peptide resin. Finally, a lysate prepared by evenly mixing TFA, TIS, EDT, TA, water and anisole was used to treat the peptide resin. The side chain protective groups were removed, and the resin cracked at the same time, to give a crude peptide product (SEQ ID NO: 12): Ala-Val-Ser-Glu-His-Gln-Leu-Leu-His-Asp-Lys-Gly-Lys-Ser-Ile-Gln-Asp-Leu-Arg-Arg-Arg-Phe-Phe-Leu-His-His-Leu-Ile-Ala-Glu-Ile-His-Thr-Ala-Tyr-Gly-Phe-Gly-Gly.

The polypeptide crude product was purified by reverse phase preparative high pressure liquid phase chromatography (HPLC), the detection wavelength was 220 nm, the mobile phase A was 0.1% TFA, and the mobile phase B was acetonitrile. The fractions containing pure products were combined, and freeze-dried to give the polypeptide product. The purity detected by HPLC was 94.8%. The obtained end product was identified by MALDI/TOF-MS.

MALDI/TOF-MS(ESI) (ESI): m/z 4450.12 [M+H]⁺.

According to QE identification and analysis, the sequence of the obtained polypeptide compound was as that shown as SEQ ID NO: 12.

Example 5:
(the polypeptide sequence shown as SEQ ID No. 13)
Preparation of Ala-Val-Ser-Glu-His-Gln-Leu-Leu-
His-Asp-Lys-Gly-Lys-Ser-Ile-Gln-Asp-Leu-Arg-Arg-
Arg-Phe-Phe-Leu-His-His-Leu-Ile-Aib-Glu-Ile-His-
Thr-Ala-Tyr-Arg-Phe-Gly-Gly

The process as shown in Example 2 was adopted. 2-CTA resin having a degree of substitution of 0.93 mmol/g was used. The resin was firstly swelled to prepare 2-CTA resin into CTC resin. Then the side chain-protected amino acids Fmoc-Gly-OH, Fmoc-Gly-OH, Fmoc-Phe-OH, Fmoc-Arg(pbf)-OH, Fmoc-Tyr(t-Bu)-OH, Fmoc-Ala-OH, Fmoc-Thr(t-Bu)-OH, Fmoc-His(trt)-OH, Fmoc-Ile-OH, Fmoc-Glu(Ot-Bu)-OH, Fmoc-Aib-OH, Fmoc-Ile-OH, Fmoc-Leu-OH, Fmoc-His(trt)-OH, Fmoc-His(trt)-OH, Fmoc-Leu-OH, Fmoc-Phe-OH, Fmoc-Phe-OH, Fmoc-Arg(pbf)-OH, Fmoc-Arg(pbf)-OH, Fmoc-Arg(pbf)-OH, Fmoc-Leu-OH, Fmoc-Asp(Ot-Bu)-OH, Fmoc-Gln(trt)-OH, Fmoc-Ile-OH, Fmoc-Ser(t-Bu)-OH, Fmoc-Lys(Boc)-OH, Fmoc-Gly-OH, Fmoc-Lys(Boc)-OH, Fmoc-Asp(Ot-Bu)-OH, Fmoc-His(trt)-OH, Fmoc-Leu-OH, Fmoc-Leu-OH, Fmoc-Gln(trt)-OH, Fmoc-His(trt)-OH, Fmoc-Glu(Ot-Bu)-OH, Fmoc-Ser(t-Bu)-OH, Fmoc-Val-OH and Fmoc-Ala-OH were successively linked to give the peptide resin. Finally, a lysate prepared by evenly mixing TFA, TIS, EDT, TA, water and anisole was used to treat the peptide resin. The side chain protective groups were removed, and the resin cracked at the same time, to give a crude peptide product (SEQ ID NO: 13): Ala-Val-Ser-Glu-His-Gln-Leu-Leu-His-Asp-Lys-Gly-Lys-Ser-Ile-Gln-Asp-Leu-Arg-Arg-Arg-Phe-Phe-Leu-His-His-Leu-Ile-Aib-Glu-Ile-His-Thr-Ala-Tyr-Arg-Phe- Gly-Gly.

The polypeptide crude product was purified by reverse phase preparative high pressure liquid phase chromatography (HPLC), the detection wavelength was 220 nm, the mobile phase A was 0.1% TFA, and the mobile phase B was acetonitrile. The fractions containing pure products were combined, and freeze-dried to give the polypeptide product. The purity detected by HPLC was 94.8%. The obtained end product was identified by MALDI/TOF-MS.

MALDI/TOF-MS(ESI): m/z 4599.253 [M+H]⁺.

According to QE identification and analysis, the sequence of the obtained polypeptide compound was as that shown as SEQ ID NO: 13.

Example 6:
(the polypeptide sequence shown as SEQ ID No. 14)
Preparation of Ala-Val-Ser-Glu-His-Gln-Leu-Ile-
His-Asp-Lys-Gly-Lys-Ser-Ile-Gln-Glu-Leu-Arg-Arg-
Arg-Phe-Phe-Leu-His-His-Leu-Ile-Aib-Glu-Ile-His-
Thr-Ala-Tyr-Gly-Phe-Gly-Gly

The process as shown in Example 2 was adopted. 2-CTA resin having a degree of substitution of 0.93 mmol/g was used. The resin was firstly swelled to prepare 2-CTA resin into CTC resin. Then the side chain-protected amino acids Fmoc-Gly-OH, Fmoc-Gly-OH, Fmoc-Phe-OH, Fmoc-Gly-OH, Fmoc-Tyr(t-Bu)-OH, Fmoc-Ala-OH, Fmoc-Thr(t-Bu)-OH, Fmoc-His(trt)-OH, Fmoc-Ile-OH, Fmoc-Glu(Ot-Bu)-OH, Fmoc-Aib-OH, Fmoc-Ile-OH, Fmoc-Leu-OH, Fmoc-His(trt)-OH, Fmoc-His(trt)-OH, Fmoc-Leu-OH, Fmoc-Phe-OH, Fmoc-Phe-OH, Fmoc-Arg(pbf)-OH, Fmoc-Arg(pbf)-OH, Fmoc-Arg(pbf)-OH, Fmoc-Leu-OH, Fmoc-Glu(Ot-Bu)-OH, Fmoc-Gln(trt)-OH, Fmoc-Ile-OH, Fmoc-Ser(t-Bu)-OH, Fmoc-Lys(Boc)-OH, Fmoc-Gly-OH, Fmoc-Lys(Boc)-OH, Fmoc-Asp(Ot-Bu)-OH, Fmoc-His(trt)-OH, Fmoc-Ile-OH, Fmoc-Leu-OH, Fmoc-Gln(trt)-OH, Fmoc-His(trt)-OH, Fmoc-Glu(Ot-Bu)-OH, Fmoc-Ser(t-Bu)-OH, Fmoc-Val-OH and Fmoc-Ala-OH were successively linked to give the peptide resin. Finally, a lysate prepared by evenly mixing TFA, TIS, EDT, TA, water and anisole was used to treat the peptide resin. The side chain protective groups were removed, and the resin cracked at the same time, to give a crude peptide product (SEQ ID NO: 14): Ala-Val-Ser-Glu-His-Gln-Leu-Ile-His-Asp-Lys-Gly-Lys-Ser-Ile-Gln-Glu-Leu-Arg-Arg-Arg-Phe-Phe-Leu-His-His-Leu-Ile-Aib-Glu-Ile-His-Thr-Ala-Tyr-Gly-Phe-Gly-Gly.

The polypeptide crude product was purified by reverse phase preparative high pressure liquid phase chromatography (HPLC), the detection wavelength was 220 nm, the mobile phase A was 0.1% TFA, and the mobile phase B was acetonitrile. The fractions containing pure products were combined, and freeze-dried to give the polypeptide product. The purity detected by HPLC was 94.8%. The obtained end product was identified by MALDI/TOF-MS.

MALDI/TOF-MS(ESI): m/z 4514.15 [M+H]⁺.

According to QE identification and analysis, the sequence of the obtained polypeptide compound was as that shown as SEQ ID NO: 14.

Example 7:
(the polypeptide sequence shown as SEQ ID No. 15)
Preparation of Ala-Val-Ser-Glu-His-Gln-Leu-Ile-
His-Asp-Lys-Gly-Lys-Ser-Ile-Gln-Glu-Leu-Arg-Arg-
Arg-Phe-Phe-Leu-His-His-Leu-Leu-Ala-Glu-Ile-His-
Thr-Ala-Tyr-Gly-Phe-Gly-Gly

The process as shown in Example 2 was adopted. 2-CTA resin having a degree of substitution of 0.93 mmol/g was used. The resin was firstly swelled to prepare 2-CTA resin into CTC resin. Then the side chain-protected amino acids Fmoc-Gly-OH, Fmoc-Gly-OH, Fmoc-Phe-OH, Fmoc-Gly-OH, Fmoc-Tyr(t-Bu)-OH, Fmoc-Ala-OH, Fmoc-Thr(t-Bu)-OH, Fmoc-His(trt)-OH, Fmoc-Ile-OH, Fmoc-Glu(Ot-Bu)-OH, Fmoc-Ala-OH, Fmoc-Leu-OH, Fmoc-Leu-OH, Fmoc-His(trt)-OH, Fmoc-His(trt)-OH, Fmoc-Leu-OH, Fmoc-Phe-OH, Fmoc-Phe-OH, Fmoc-Arg(pbf)-OH, Fmoc-Arg(pbf)-OH, Fmoc-Arg(pbf)-OH, Fmoc-Leu-OH, Fmoc-Glu(Ot-Bu)-OH, Fmoc-Gln(trt)-OH, Fmoc-Ile-OH, Fmoc-Ser(t-Bu)-OH, Fmoc-Lys(Boc)-OH, Fmoc-Gly-OH, Fmoc-Lys(Boc)-OH, Fmoc-Asp(Ot-Bu)-OH, Fmoc-His(trt)-OH, Fmoc-Ile-OH, Fmoc-Leu-OH, Fmoc-Gln(trt)-OH, Fmoc-His(trt)-OH, Fmoc-Glu(Ot-Bu)-OH, Fmoc-Ser(t-Bu)-OH, Fmoc-Val-OH and Fmoc-Ala-OH were successively linked to give the peptide resin. Finally, a lysate prepared by evenly mixing TFA, TIS, EDT, TA, water and anisole was used to treat the peptide resin. The side chain protective groups were removed, and the resin cracked at the same time, to give a crude peptide product (SEQ ID NO: 15): Ala-Val-Ser-Glu-His-Gln-Leu-Ile-His-Asp-Lys-Gly-Lys-Ser-Ile-Gln-Glu-Leu-Arg-Arg-Arg-Phe-Phe-Leu-His-His-Leu-Leu-Ala-Glu-Ile-His-Thr-Ala-Tyr-Gly-Phe-Gly-Gly.

The polypeptide crude product was purified by revers phase preparative high pressure liquid phase chromatography (HPLC), the detection wavelength was 220 nm, the mobile phase A was 0.1% TFA, and the mobile phase B was acetonitrile. The fractions containing pure products were combined, and freeze-dried to give the polypeptide product. The purity detected by HPLC was 94.8%. The obtained end product was identified by MALDI/TOF-MS.

MALDI/TOF-MS(ESI): m/z 4514.15 [M+H]⁺.

According to QE identification and analysis, the sequence of the obtained polypeptide compound was as that shown as SEQ ID NO: 15.

### Examples of Effect

### (I) Research on Osteoporosis Therapeutic Effect of the Compound in the Present Disclosure on Ovary Removed SD Rats

Experimental method: female SD rats aged around 22 weeks were used in the experiment. Feeding conditions: the animal room had a temperature of 21±5° C., and a relative humidity of 35±10%; and the animal room was exposed to light for 12 h and darkness for 12 h in each day. The animals had free access to water. The SD rats were subjected to ovariectomy (OVX), then fed for another 3 months to be induced to osteoporosis model. The rats were grouped according to bone mineral density of thigh bone: \textcircled{1} Sham group: normal saline of the same volume was subcutaneously administered; \textcircled{2} OVX group (model group): normal saline of the same volume was subcutaneously administered; \textcircled{3} 5 μg dosage group of positive drug abaloptide (Aba-5): 5 μg/kg abaloptide (Aba) was subcutaneously administered. Administration groups of three dosages were set for each test compound. 2.5 μg/kg dosage group of the test compound: 2.5 μg/kg test compound was subcutaneously administered. 5 μg/kg dosage group of the test compound: 5 μg/kg test compound was subcutaneously administered. 10 μg/kg dosage group of the test compound: 10 μg/kg test compound was subcutaneously administered. The administration was carried out 5 times a week, successively lasted for 25 weeks. The test compounds were polypeptide compounds in examples 1-5 (as shown in SEQ ID NO:9-SEQ ID NO:13). Abaloptide was a drug for treating post-menopausal osteoporosis in the market.

Detection method: times and contents of detection indicators were shown hereinafter.

1) Bone mineral density (BMD) was detected with a dual-energy X-ray bone densitometry (DXA) before model establishment (−13 w), before administration (0 w), and 6 w, 12 w, 25 w, 37 w and 49 w after administration.

2) Blood was collected from eye orbit before administration (0 w) and 25 w after administration, and a blood routine examination was carried out with whole blood.

3) 25 w after administration, the serum was kept, and bone turnover markers (CTx (β-CTX, serum), OC (serum) and serum procollagen type 1 N-terminal propeptide (PINP)) were detected, and calcium, phosphorus and alkaline phosphatase (ALP) were detected.

4) After administration, thigh bones of both sides and lumbar vertebra of rats were collected, and subjected to MicroCT, histomorphology detection and biomechanics detections.

Statistical analysis of data: the data were treated with SPSS 20 software. The data in line with normality test were subjected to one-way analysis of variance; and the data not in line with normality test were subjected to rank sum test. The data were represented by mean value±standard error.

### Experimental Results

(1) The bone mineral densities of thigh and lumbar vertebra were counted, and percentages of density changes of thigh bone and lumbar vertebra at each detection time point were calculated, shown in Table 1.

Percentage of bone mineral density change=(bone mineral density at the detection time point−bone density before administration)/bone density before administration*100%

TABLE 1
Bone mineral density and percentages of change of rats in each group

| | | 0 w Bone mineral density | | 25 w Bone mineral density | | | |
|---|---|---|---|---|---|---|---|
| | | (g/cm²) | | (g/cm²) | | | |
| | Dosage | | lumbar | | lumbar | 0-25 w percentage of change % | |
| Group | (μg/kg) | thigh bone | vertebra | thigh bone | vertebra | thigh bone | lumbar vertebra |
| Sham | — | 0.26 ± 0 | 0.28 ± 0.01 | 0.28 ± 0 | 0.29 ± 0.01 | 7.6 ± 2.03 | 4.73 ± 3.19 |
| Model/OVX | — | 0.23 ± 0*** | 0.25 ± 0*** | 0.22 ± 0*** | 0.21 ± 0*** | −3.77 ± 1.19*** | −14.41 ± 1.58*** |
| OVX + Aba | 5 | 0.23 ± 0*** | 0.24 ± 0*** | 0.3 ± 0*### | 0.3 ± 0### | 30.28 ± 2***### | 21.66 ± 2.12***### |
| | 2.5 | 0.23 ± 0.01** | 0.21 ± 0.00*** | 0.28 ± 0.01* | 0.23 ± 0.01* | 30.75 ± 3.69# | 2.82 ± 4.46 |
| OVX + | 5 | 0.23 ± 0.01** | 0.22 ± 0.00*** | 0.30 ± 0.01## | 0.27 ± -0.01 | 37.67 ± 2.70## | 24.82 ± 4.93## |
| Example 1 | 10 | 0.25 ± 0.01** | 0.22 ± 0.00*** | 0.31 ± 0.01# | 0.27 ± 0.01# | 38.15 ± 4.87# | 24.91 ± 7.23# |
| | 2.5 | 0.23 ± 0*** | 0.24 ± 0*** | 0.28 ± 0### | 0.29 ± 0.01### | 22.21 ± 1.54***### | 18.49 ± 2.13### |
| OVX + | 5 | 0.23 ± 0*** | 0.24 ± 0*** | 0.3 ± 0### | 0.31 ± 0.01### | 29.17 ± 1.86***### | 29.61 ± 2.26***### |
| Example 2 | 10 | 0.23 ± 0*** | 0.24 ± 0.01*** | 0.31 ± 0***### | 0.31 ± 0.01### | 33.68 ± 1.84***### | 30.94 ± 2.24***### |
| | 2.5 | 0.23 ± 0*** | 0.24 ± 0*** | 0.26 ± 0### | 0.27 ± 0### | 14.03 ± 1.81***### | 12.4 ± 2.2## |
| OVX + | 5 | 0.23 ± 0*** | 0.24 ± 0*** | 0.29 ± 0.02### | 0.29 ± 0.01### | 27.19 ± 1.33***### | 20.41 ± 2.56### |
| Example 3 | 10 | 0.23 ± 0*** | 0.24 ± 0.01*** | 0.29 ± 0### | 0.29 ± 0.01### | 28.7 ± 1.6***### | 22.46 ± 2.25### |
| | 2.5 | 0.23 ± 0*** | 0.24 ± 0*** | 0.27 ± 0### | 0.28 ± 0.01### | 17.23 ± 1.4***### | 14.05 ± 2.79*### |
| OVX + | 5 | 0.23 ± 0*** | 0.24 ± 0*** | 0.28 ± 0### | 0.31 ± 0.01### | 31.61 ± 1.54***### | 25.79 ± 2.06***### |
| Example 4 | 10 | 0.23 ± 0*** | 0.24 ± 0.01*** | 0.29 ± 0*### | 0.32 ± 0.01### | 35.37 ± 1.94***### | 24.06 ± 2.19***### |
| | 2.5 | 0.23 ± 0.01** | 0.21 ± 0.00*** | 0.3 ± 0.01# | 0.26 ± 0.01* | 40.87 ± 4.24# | 18.36 ± 4.34 |
| OVX + | 5 | 0.23 ± 0.01** | 0.22 ± 0.00*** | 0.32 ± 0.01## | 0.29 ± 0.02## | 46.29 ± 2.42## | 34.29 ± 7.15## |
| Example 5 | 10 | 0.25 ± 0.01** | 0.22 ± 0.00*** | 0.3 ± 0.01# | 0.27 ± 0.01# | 35.57 ± 3.17# | 32.67 ± 5.38# |

Note:
compared with Sham group, *represented for P < 0.05, **represented for P < 0.01, and ***represented for P < 0.001;
compared with OVX group, #represented for P < 0.05, ##represented for P < 0.01, and ###represented for P < 0.001; and
compared with Aba-5 μ/kg group, & represented for P < 0.05, && represented for P < 0.01, and &&& represented for P < 0.001.

Result and discussion: on the base of Table 1, compared with OVX model group, the test compound groups of examples 1-5 were administered at dosages of 2.5 μg/kg, 5 μg/kg and 10 μg/kg for 25 weeks, bone mineral densities of thigh bone and lumbar vertebra significantly increased in OVX induced rat osteoporosis model. In addition, there was a dose-response relationship between the test compounds in examples 1-5 and the increase of bone mineral density of osteoporosis rat.

Compared with sham group, the test compound groups of examples 1-5 were administered at dosages of 2.5 μg/kg, 5 μg/kg and 10 μg/kg for 25 weeks, bone mineral densities of thigh bone and lumbar vertebra of osteoporosis rat increased, and there was no significant difference in bone mineral density compared with the bone mineral density of sham group in the end. In some administration groups, the bone mineral densities of rats were even higher than that of the sham group, indicating that 25 weeks after administration, bone mineral densities of thigh bone and lumbar vertebra were already close to normal level. This demonstrated that the polypeptide compound in the present disclosure can facilitate ossification, and increase bone mineral density.

After administering the marketed drug abaloptide at a dosage of 5 μg/kg for 25 weeks, the bone mineral density of thigh bone of osteoporosis rat increased by 30.28±2%. When the test compounds 1-5 were administered at a dosage of 5 μg/kg, the bone mineral density of thigh bone of osteoporosis rat respectively increased by 37.67±2.70%, 29.17±1.86%, 27.19±1.33%, 31.61±1.54 and 46.29±2.42%. After administering the marketed drug abaloptide at a dosage of 5 μg/kg for 25 weeks, the bone mineral density of lumbar vertebra of osteoporosis rat increased by 21.66±2.12%. When the test compounds 1-5 were administered at a dosage of 5 μg/kg, the bone mineral density of lumbar vertebra of osteoporosis rat respectively increased by 24.82±4.93%, 29.61±2.26%, 20.41±2.56%, 25.79±2.06% and 34.29±7.15%.

(2) Blood routine examination results of animals in each group were counted, shown in Table 2.

TABLE 2
immunity-related indicators of peripheral
blood of rats in each group

| | | immunity-related indicators of peripheral blood | | | |
|---|---|---|---|---|---|
| | Dosage | WBC | Lymph | Gran | Gran/ |
| Group | (μg/kg) | (10⁹/L) | (10⁹/L) | (10⁹/L) | lymph |
| Sham | — | 6.32 ± | 3.48 ± | 2.57 ± | 73.79 ± |
| | | 0.38 | 0.16 | 0.25 | 6.5 |
| Model/ | — | 6.25 ± | 3.89 ± | 2.12 ± | 55.16 ± |
| OVX | | 0.38 | 0.23 | 0.16 | 2.96 |
| OVX+ | 5 | 4.31 ± | 2.72 ± | 1.45 ± | 53.27 ± |
| Aba | | 0.22*## | 0.13### | 0.09* | 2.19* |
| OVX+ | 2.5 | 6.47 ± | 3.85 ± | 2.13 ± | 74.12 ± |
| Exam- | | 1.1&& | 0.96&& | 0.44& | 0.17&& |
| ple 1 | 5 | 6.06 ± | 3.12 ± | 2.72 ± | 70.30 ± |
| | | 1.26&& | 0.99 | 0.44&&& | 0.15&& |
| | 10 | 6.98 ± | 3.4 ± | 2.17 ± | 71.22 ± |
| | | 0.88&&& | 0.58 | 0.488z | 0.11&& |
| OVX + | 2.5 | 6.13 ± | 3.63 ± | 2.27 ± | 64.17 ± |
| Exam- | | 0.32&& | 0.21&& | 0.16& | 4.328z |
| ple 2 | 5 | 6.31 ± | 3.4 ± | 2.7 ± | 75.15 ± |
| | | 0.4&& | 0.18 | 0.27&&& | 5.87#&& |
| | 10 | 6.61 ± | 3.7 ± | 2.7 ± | 67.79 ± |
| | | 0.47&&& | 0.21&& | 0.27&&& | 4.48 |
| OVX + | 2.5 | 6.65 ± | 3.68 ± | 2.36 ± | 74.77 ± |
| Exam- | | 0.63&& | 0.27&& | 0.33& | 4.59##&&& |
| ple 3 | 5 | 6.55 ± | 3.97 ± | 2.92 ± | 72.27 ± |
| | | 0.75&& | 0.28&&& | 0.43#&&& | 5.29#&& |
| | 10 | 6.12 ± | 3.7 ± | 2.03 ± | 70.35 ± |
| | | 0.47&& | 0.27&& | 0.26& | 4.41#&& |
| OVX + | 2.5 | 6.5 ± | 3.29 ± | 2.08 ± | 61.06 ± |
| Exam- | | 0.35&& | 0.24 | 0.14& | 3.2**& |
| ple 4 | 5 | 6.87 ± | 3.4 ± | 2.2 ± | 60.15 ± |
| | | 0.39&&& | 0.23 | 0.168z | 2.14**& |
| | 10 | 6.74 ± | 3.49 ± | 2.87 ± | 63.82 ± |
| | | 0.41&& | 0.28& | 0.11#&&& | 2.55*#&& |
| OVX + | 2.5 | 6.48 ± | 3.58 ± | 2.58 ± | 65.15 ± |
| Exam- | | 4.47&& | 2.41& | 1.91&& | 3.23&& |
| ple 5 | 5 | 6.34 ± | 3.8 ± | 2.18 ± | 73.15 ± |
| | | 2.03& | 1.26&& | 1.02& | 4.78#&& |
| | 10 | 6.8 ± | 3.4 ± | 2.57 ± | 66.54 ± |
| | | 1.38&& | 1.02 | 0.31&& | 2.38#&& |

Note:
WBC: white blood cell,
Lymph: lymphocyte,
Gran: neutrophile granulocyte;
compared with Sham group,
*represented for P <0.05,
**represented for P <0.01, and
***represented for P<0.001;
compared with OVX group,
#represented for P <0.05,
##represented for P <0.01,
###represented for P <0.001;
and compared with Aba-5 μ/kg group,
&represented for P <0.05,
&&represented for P <0.01, and
&&&represented for P <0.001.

Results and discussion: on the basis of Table 2, it could be concluded that compared with Sham group, the number of white blood cells, lymphocytes and neutrophile granulocytes of osteoporosis rat in OVX group did not significantly change, indicating that karyocyte level in peripheral blood of osteoporosis rat kept normal.

In abaloptide control group, after administering for 25 weeks, the number of white blood cell significantly decreased compared with OVX osteoporosis model control group and Sham control group, and the number of lymphocytes significantly decreased compared with Sham control group, and the number of neutrophile granulocyte significantly decreased compared with OVX group.

Compared with OVX model group and Sham control group, after administering the test compounds in examples 1-5 at dosages of 2.5 μg/kg, 5 μg/kg and 10 μg/kg for 25 weeks, the number of white blood cells, lymphocytes and neutrophile granulocyte kept normal, indicating that the compound of the present disclosure stabilized the number of karyocyte in peripheral blood while facilitating ossification and improving bone mineral density of osteoporosis rat.

Compared with 5 μg/kg administration group of abaloptide, after administering the test compounds in examples 1-5 at dosages of 2.5 μg/kg, 5 μg/kg and 10 μg/kg for 25 weeks, the number of white blood cells, lymphocytes and neutrophile granulocyte in peripheral blood was significantly higher than that of abaloptide group. During the administration period of abaloptide, mononuclear cell, lymphocyte and white blood cells in peripheral blood significantly decreased, while the compounds in the present disclosure significantly stablized the karyocyte level in peripheral blood cells, and overcame the adverse effects of abaloptide.

### (II) Research on Retinoic Acid-Induced Osteoporosis Therapeutic Effect of the Compound in the Present Disclosure

Experimental method: SD rats were administered by gavage to induce a rat osteoporosis model. After model establishment, the rats were randomly grouped according to bone mineral densities of thigh bone: \textcircled{1} vehicle group (Control group): normal saline was subcutaneously administered in an equal volume to the test compound, and soybean oil was administered by gavage in an equal volume to retinoic acid; \textcircled{2} Model group: normal saline of the same volume was subcutaneously administered; and administered with retinoic acid (RA) by gavage every other day to maintain osteoporosis state; \textcircled{3} 20 μg dosage group of positive drug abaloptide (Aba-20): 10 μg/kg abaloptide was subcutaneously administered, and retinoic acid was administered by gavage every other day. Administration groups of three dosages were set for each test compound. −10 μg/kg dosage group of the test compound: 10 μg/kg test compound was subcutaneously administered, and retinoic acid was administered by gavage every other day. −20 μg/kg dosage group of the test compound: 20 μg/kg test compound was subcutaneously administered, and retinoic acid was administered by gavage every other day. −40 μg/kg dosage group of the test compound: 40 μg/kg test compound was subcutaneously administered, and retinoic acid was administered by gavage every other day. The administration was carried out 5 times a week, with normal saline as the diluent of test drug, and the administration successively lasted for 12 weeks. The induction dosage of retinoic acid was about 80 mg/kg and the maintenance dosage after model establishment was about 30 mg/Kg, and solvent of inducing agent was soybean oil.

Times and contents of detection indicators were shown hereinafter.

1) Bone mineral density (BMD) was detected with a dual-energy X-ray bone densitometry (DXA) before administration (0 w), and 12 w after administration.

2) Blood was collected from eye orbit before administration (0 w) and 12 w after administration, the serum was removed to carry out a detection of calcium, phosphorus and alkaline phosphatase (ALP); and the other part of whole blood was subjected to a blood routine examination.

3) 12 w after administration, the serum of blood collected from eye orbit was subjected to detection of bone turnover markers (CTx (β-CTX, serum), OC (serum) and serum procollagen type 1 N-terminal propeptide (PINP)).

After administration completed, tibia on the left was used to prepare a bone marrow smear. The thigh bone on the left was subjected to three-point bending test, and the thigh bone on the right was subjected to CT scan and was used to prepare a pathological section.

Statistical analysis of data: the data were treated with SPSS 20 software. The data in line with normality test were subjected to one-way analysis of variance; and the data not in line with normality test were subjected to rank sum test. The data were represented by mean value±standard error.

### Experimental Results

(1) Influence of the Compound on Bone Marrow Karyocyte of Retinoic Acid-Induced Osteoporosis Rats

The number of karyocyte in bone marrow cavity of retinoic acid-induced osteoporosis rats was counted, shown in Table 3.

TABLE 3
number of bone marrow karyocyte
12 weeks after administration

| | Dosage | Number of cells |
|---|---|---|
| Group | (μg/kg) | (*10⁵) |
| Control | — | 235.35 ± 26.08 |
| Model/RA | — | 190.44 ± 29.83 |
| RA + Aba | 20 | 112.21 ± 13.85***# |
| RA + | 10 | 187.66 ± 15.78&& |
| Example 1 | 20 | 195.18 ± 15.22& |
| | 40 | 189.5 ± 27.29& |
| RA + | 10 | 210.43 ± 23.99&& |
| Example 2 | 20 | 205.81 ± 14.39& |
| | 40 | 187.5 ± 27.29& |
| RA + | 10 | 186.57 ± 16.89& |
| Example 3 | 20 | 201.82 ± 12.93& |
| | 40 | 193.15 ± 20.31& |
| RA + | 10 | 198.21 ± 13.76& |
| Example 4 | 20 | 201.82 ± 12.93& |
| | 40 | 203.45 ± 27.29& |
| RA + | 10 | 206.11 ± 20.32&& |
| Example 5 | 20 | 200.33 ± 9.93& |
| | 40 | 210.00 ± 26.14&& |

Note:
Aba: positive drug abaloptide;
RA: retinoic acid; compared with Control group,
***represented for p <0.001;
compared with RA group,
#represented for p <0.05;
compared with Aba-20 μg/kg group,
&& represented for p <0.01;
and compared with Aba-20 μg/kg group,
& represented for p <0.05.

Results and discussion: after administering for 12 w, compared with Control group, the number of karyocyte in bone marrow cavity significantly decreased in 20 μg/kg group of abaloptide (P<0.001); compared with model group, the number of karyocyte in bone marrow cavity significantly decreased in 20 μg/kg group of abaloptide (P<0.05). Retinoic acid-induced osteoporosis rat did not have bone marrow suppression, and abaloptide had obvious inhibiting effects on bone marrow of normal rats and osteoporosis rats.

Compared with 20 μg/kg dosage group of abaloptide, the numbers of karyocyte in 10 and 20 μg/kg dosage groups of test compounds 1-5 significantly increased (P<0.05). The numbers of bone marrow karyocyte in administration groups of test compounds 1-5 were close to normal level (there was no significant difference compared with control group). The compounds in the present disclosure stablized the number of bone marrow karyocyte.

(2) Influence of the Compound on Microstructure of Bone of Retinoic Acid-Induced Osteoporosis Rats

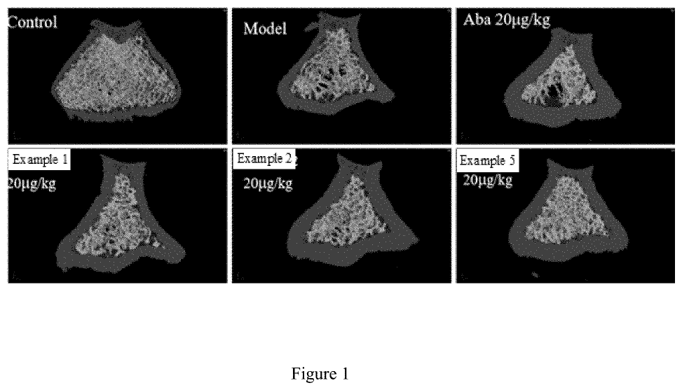

After the experiment was completed, thigh bone on the right side of rats were collected, and subjected to CT detection, and bone surface area/bone volume ratio (BV/TV), the number of trabeculae (TbN) and trabeculae spacing (TbSp) were calculated, and the results were shown in Table 4. An example was selected from each of Control group, Model group, abaloptide group, 20 μg/kg dosage group of Example 1, 20 μg/kg dosage group of Example 2 and 20 μg/kg dosage group of Example 5, and the scanned figures by micron X-ray 3D imaging system of trochlea of their thigh bone were shown in FIG. 1.

TABLE 4
Effect of the compound on the bone microstructure
of rats with retinoic acid-induced osteoporosis

| | | Indicators of CT | | |
|---|---|---|---|---|
| | Dosage | | TbN | TbSp |
| Group | μg/kg | BV/TV | (mm⁻¹) | (mm) |
| Control | — | 0.32 ± | 5.22 ± | 0.13 ± |
| | | 0.02 | 0.19 | 0.01 |
| Model/RA | — | 0.15 ± | 2.9 ± | 0.32 ± |
| | | 0.02^{**} | 0.43^{***} | 0.05^{***} |
| RA + Aba | 20 | 0.28 ± | 3.4 ± | 0.22 ± |
| | | 0.02 | 0.2^{***} | 0.02 |
| RA + | 10 | 0.34 ± | 4.24 ± | 0.17 ± |
| Example 1 | | 0.03^{###} | 0.16^{#} | 0.02^{##} |
| | 20 | 0.32 ± | 4.01 ± | 0.19 ± |
| | | 0.01^{##} | 0.34 | 0.03^{##} |
| | 40 | 0.41 ± | 4.25 ± | 0.14 ± |
| | | 0.02^{###} | 0.15^{#} | 0.01^{###} |
| RA + | 10 | 0.35 ± | 4.10 ± | 0.17 ± |
| Example 2 | | 0.04^{###} | 0.28 | 0.03^{##} |
| | 20 | 0.32 ± | 4.06 ± | 0.18 ± |
| | | 0.03^{##} | 0.36 | 0.04^{##} |
| | 40 | 0.40 ± | 4.38 ± | 0.15 ± |
| | | 0.04^{###} | 0.17^{#} | 0.04^{##} |
| RA + | 10 | 0.33 ± | 4.17 ± | 0.20 ± |
| Example 3 | | 0.01^{###} | 0.28^{#} | 0.04^{#} |
| | 20 | 0.30 ± | 4.21 ± | 0.17 ± |
| | | 0.02^{##} | 0.16^{#} | 0.04^{##} |
| | 40 | 0.39 ± | 4.22 ± | 0.15 ± |
| | | 0.05^{##} | 0.37^{#} | 0.02^{##} |
| RA + | 10 | 0.35 ± | 4.23 ± | 0.17 ± |
| Example 4 | | 0.01^{###} | 0.21 | 0.03^{##} |
| | 20 | 0.37 ± | 4.56 ± | 0.18 ± |
| | | 0.02^{##} | 0.33^{#} | 0.04^{##} |
| | 40 | 0.40 ± | 4.58 ± | 0.15 ± |
| | | 0.03^{###} | 0.15^{#} | 0.04^{##} |
| RA + | 10 | 0.33 ± | 4.19 ± | 0.16 ± |
| Example 5 | | 0.03^{###} | 0.28^{#} | 0.03^{##} |
| | 20 | 0.36 ± | 4.42 ± | 0.15 ± |
| | | 0.02^{##} | 0.16^{#} | 0.06^{##} |
| | 40 | 0.39 ± | 4.48 ± | 0. 14 ± |
| | | 0.02^{###} | 0.09^{#} | 0.03^{##} |

Note: compared with Control group,
*represented for p <0.05,
**represented for p <0.01,
***represented for p <0.001;
compared with RA group,
#represented for p <0.05,
##represented for p <0.01, and
###represented for p <0.001;
compared with Aba-20 μg/kg group,
& represented for p <0.05; and
n = 6-8.

Results and discussion: it can be concluded from Table 4 that, after administering for 12 w, compared with Control group, the bone volume/total volume BV/TV (P<0.01) and the number of trabeculae TbN (P<0.001) of RA group significantly decreased, and the trabeculae spacing TbSp (P<0.001) significantly increased, indicating that microstructure of bone tissue of rats in Model (RA) group was seriously damaged.

Compared with model group/RA, the bone volume/total volume BV/TV and the number of trabeculae TbN of each dosage group of the test compounds 1-5 significantly increased, and the trabeculae spacing TbSp (P<0.001) significantly decreased, indicating that bone mass of thigh bone of rats increased and microstructure damage of bone tissue was repaired after administering the test compounds of the present disclosure.

Under the same dosage (20 μg/kg), the test compounds 1-5 were obviously superior to abaloptide in aspects of improving bone surface area/total volume ratio (BV/TV), number of trabeculae (TbN) and trabeculae spacing (TbSp), showing that the compounds of the present disclosure were superior to abaloptide in treating high-turnover osteoporosis.

(3) Influence of the Compound on Biomechanics of Retinoic Acid-Induced Osteoporosis Rats

After the experiment, thigh bones on the left of rats were collected, subjected to three-point mechanical test, and the results of experiments were shown in Table 5.

TABLE 5
Influence of the compound on three-point
mechanical test of osteoporosis rats

| | | Peak load of |
|---|---|---|
| | Dosage | three-point |
| Group | (μg/kg) | mechanics (N) |
| Control | — | 144.08 ± |
| | | 7.87 |
| RA(Model) | — | 88.05 ± |
| | | 7.1*** |
| RA + Aba | 20 | 119.57 ± |
| | | 6.32# |
| RA + | 10 | 98.11 ± |
| Example 1 | | 5.43** |
| | 20 | 123.54 ± |
| | | 3.95# |
| | 40 | 130.38 ± |
| | | 8.54## |
| RA + | 10 | 97.87 ± |
| Example 2 | | 8.71** |
| | 20 | 122.02 ± |
| | | 6.65# |
| | 40 | 133.38 ± |
| | | 9.70## |
| RA + | 10 | 97.65 ± |
| Example 3 | | 8.53** |
| | 20 | 126.54 ± |
| | | 6.76# |
| | 40 | 131.74 ± |
| | | 5.89## |
| RA + | 10 | 99.01 ± |
| Example 4 | | 9.12** |
| | 20 | 125.43 ± |
| | | 7.44# |
| | 40 | 136.96 ± |
| | | 8.52## |
| RA + | 10 | 98.47 ± |
| Example 5 | | 8.84** |
| | 20 | 127.02 ± |
| | | 8.63# |
| | 40 | 132.38 ± |
| | | 7.32## |

Note:
Aba: positive drug abaloptide;
RA: inducing agent retinoic acid; compared with Control group,
**represented for p <0.01, and
***represented for p <0.001; compared with RA group,
#represented for p <0.05, and
##represented for p <0.01; and
n = 5-14.

Results and discussion: after administering for 12 w, thigh bone three-point mechanical test was carried out. Compared with Control group, the peak load of Model group significantly decreased (P<0.001), indicating that the peak load of thigh bone of retinoic acide-induced osteoporosis rats significantly decreased.

Compared with Model group, peak loads of thigh bone of rats in the 20 μg/kg, 40 μg/kg dosage groups of test compound significantly increased (P<0.05). Peak load of 20 μg/kg dosage group of abaloptide did not significantly increase compared with Model group. In the aspect of improving peak load of thigh bone of osteoporosis rat, the compounds of the present disclosure were superior to abaloptide.

In summary, compared with abaloptide, the compounds in the present disclosure can significantly stablize karyocyte level in both bone marrow and peripheral blood. The compounds in the present disclosure can increase the peak load of retinoic acid-induced osteoporosis rat, and the effect is better than that of abaloptide. The compounds of the present disclosure can also improve microstructure of bone, specifically including improving bone surface area/total volume ratio (BV/TV), trabeculae number (TbN) and trabecular spacing, and the effect is better than the marketed drug abaloptide.

The present disclosure also relates to the following embodiments:

1. An active polypeptide compound, which has a structure represented by following Formula (Ia) or Formula (Ib), or is a pharmaceutically acceptable salt thereof,

Y-ID-X Formula (Ia), or

X-ID-Y Formula (Ib),

wherein,

Y is a PTH/PTHrP receptor agonist or an osteoclast inhibitor;

ID is a peptide bond or a linker in the molecule, which links X to Y; and

X is an osteogenic growth peptide receptor agonist, a bone marrow mesenchymal stem cell irritant or a hematopoietic stem cell irritant.

2. The active polypeptide compound according to embodiment 1, wherein Y is M-CSF antagonist, RANKL inhibitor, RANKL antibody, MMP inhibitor, calcitonin, parathyroid hormone or parathyroid hormone-related protein.

3. The active polypeptide compound according to embodiment 1 or 2, wherein Y is a peptide chain having an amino acid sequence as shown in Formula (II):

A₁-Val-Ser-Glu-His-Gln-Leu-A₈-His-Asp-Lys-Gly-Lys-Ser-Ile-Gln-A₁₇-Leu-Arg-Arg-Arg-A₂₂-A₂₃-Leu-A₂₅-A₂₆-Leu-A₂₈-A₂₉-A₃₀-A₃₁-His-Thr-Ala Formula (II);

wherein, A₁ is Ala, Val, Leu or Ile;

A₈ is Leu or Ile;

A₁₇ is Asp or Glu;

A₂₂ is Glu, Asp or Phe;

A₂₃ is Leu, Ile or Phe;

A₂₅ is Glu, Asp or His;

A₂₆ is Lys, His or Arg;

A₂₈ is Leu, Ile or Val;

A₂₉ is Ala, (N-Me)Ala or Aib;

A₃₀ is Lys or Glu;

A₃₁ is Leu or Ile;

the amino terminal of the peptide chain Y is free or chemically modified, and the carboxyl terminal of the peptide chain Y is free or chemically modified.

4. The active polypeptide compound according to any one of embodiments 1-3, wherein X is a hematopoietic stem cell irritant, a hematopoietic growth factor, a platelet colony-stimulating factor, a granulocyte colony-stimulating factor, erythropoietin, interleukin 3 or recombinant human interleukin 11.

5. The active polypeptide compound according to any one of embodiments 1-4, wherein X is a peptide chain having an amino acid sequence as shown in Formula (IIIa) or Formula (IIIb):

Tyr-(Arg)m-(Gly)ₙ-Phe-Gly-Gly Formula (IIIa)

Gly-Gly-Phe-(Gly)ₙ-(Arg)ₘ-Tyr Formula (IIIb);

wherein, m and n are independently 0, 1 or 2; and

the amino terminal of the peptide chain X is free or chemically modified, and the carboxyl terminal of the peptide chain X is free or chemically modified.

6. The active polypeptide compound according to embodiment 5, wherein X is a peptide chain consisting of 5-6 amino acids which has an amino acid sequence as shown in one of the following SEQ ID NO:1-SEQ ID NO:8:

(SEQ ID NO: 1)
Tyr-Gly-Phe-Gly-Gly
(SEQ ID NO: 2)
Tyr-Arg-Phe-Gly-Gly
(SEQ ID NO: 3)
Tyr-Arg-Gly-Phe-Gly-Gly
(SEQ ID NO: 4)
Tyr-Pro-Phe-Gly-Gly
(SEQ ID NO: 5)
Gly-Gly-Phe-Gly-Tyr
(SEQ ID NO: 6)
Gly-Gly-Phe-Arg-Tyr
(SEQ ID NO: 7)
Gly-Gly-Phe-Gly-Arg-Tyr
(SEQ ID NO: 8)
Gly-Gly-Phe-Pro-Tyr.

7. The active polypeptide compound according to any one of embodiments 1-6, wherein ID is a linker between X and Y; the linker is an amino-substituted C₁₋₈ alkyl carboxylic acid, a polyethylene glycol polymer chain or a peptide segment consisting of 1-10 amino acids, and the amino acids in the peptide segment is selected from the group consisting of proline, arginine, alanine, threonine, glutamic acid, aspartic acid, lysine, glutamine, asparagine and glycine.

8. The active polypeptide compound according to embodiment 7, wherein the linker is one of the following linkers:

(1) (Gly-Ser)ₚ, wherein p is 1, 2, 3, 4 or 5;

(2) (Gly-Gly-Gly-Gly-Ser)ₜ, wherein t is 1, 2 or 3;

(3) Ala-Glu-Ala-Ala-Ala-Lys-Ala;

(4) 4-aminobutyric acid or 6-aminocaproic acid; and

(5) (PEG)_{q}, wherein q is 1, 2, 3, 4 or 5.

9. The active polypeptide compound according to any one of embodiments 1-8, which has a structure as shown in Formula (IV), or is a pharmaceutically acceptable salt of the compound shown as in Formula (IV):

A₁-Val-Ser-Glu-His-Gln-Leu-A₈-His-Asp-Lys-Gly-Lys-Ser-Ile-Gln-A₁₇-Leu-Arg-Arg-Arg-A₂₂-A₂₃-Leu-A₂₅-A₂₆-Leu-A₂₈-A₂₉-A₃₀-A₃₁-His-Thr-Ala-A₃₅ Formula (IV),

wherein, A₁ is Ala, Val, Leu or Ile;

A₈ is Leu or Ile;

A₁₇ is Asp or Glu;

A₂₂ is Glu, Asp or Phe;

A₂₃ is Leu, Ile or Phe;

A₂₅ is Glu, Asp or His;

A₂₆ is Lys, His or Arg;

A₂₈ is Leu, Ile or Val;

A₂₉ is Ala, (N-Me)Ala or Aib;

A₃₀ is Lys or Glu;

A₃₁ is Leu or Ile; and

A₃₅ has a peptide chain of the amino acid sequence as shown in Formula (IIIa) or (IIIb):

Tyr-(Arg)ₘ-(Gly)ₙ-Phe-Gly-Gly Formula (IIIa),

Gly-Gly-Phe-(Gly)ₙ-(Arg)ₘ-Tyr Formula (IIIb),

wherein, m and n are independently 0, 1 or 2; and

the amino terminal of the amino acids shown by A₁ is free or chemically modified, and the carboxyl terminal of the peptide chain A₃₅ is free or chemically modified.

10. The active polypeptide compound according to embodiment 9, wherein the chemical modifications of the amino terminal include acylation, sulfonylation, alkylation and PEG modification; and the chemical modifications of the carboxyl terminal include amidation, sulfonylation and PEG modification.

11. The active polypeptide compound according to embodiment 10, wherein the chemical modification of amino terminal is acetylation, benzoylation or sulfonylation of amino; the alkylation of amino terminal is C₁₋₆ alkylation or aromatic alkylation; the chemical modification of carboxylic terminal is that the OH in the carboxyl is substituted by NH₂ or sulfamide, or the OH in the carboxyl links to the functionalized PEG molecule.

12. The active polypeptide compound according to any one of embodiments 1-11, which is one of the compounds in the following SEQ ID NO:9-SEQ ID NO:15, or a pharmaceutically acceptable salt thereof:

(1)
SEQ ID NO: 9
Ala-Val-Ser-Glu-His-Gln-Leu-Leu-His-Asp-Lys-Gly-
Lys-Ser-Ile-Gln-Asp-Leu-Arg-Arg-Arg-Glu-Leu-Leu-
Glu-Lys-Leu-Leu-(N-Me)Ala-Lys-Leu-His-Thr-Ala-
Tyr-Gly-Phe-Gly-Gly;
(2)
SEQ ID NO: 10
Ala-Val-Ser-Glu-His-Gln-Leu-Leu-His-Asp-Lys-Gly-
Lys-Ser-Ile-Gln-Asp-Leu-Arg-Arg-Arg-Glu-Leu-Leu-
Glu-Lys-Leu-Leu-Aib-Lys-Leu-His-Thr-Ala-Tyr-Gly-
Phe-Gly-Gly;
(3)
SEQ ID NO: 11
Ala-Val-Ser-Glu-His-Gln-Leu-Leu-His-Asp-Lys-Gly-
Lys-Ser-Ile-Gln-Asp-Leu-Arg-Arg-Arg-Glu-Leu-Leu-
Glu-Lys-Leu-Leu-Ala-Lys-Leu-His-Thr-Ala-Tyr-Arg-
Gly-Phe-Gly-Gly;
(4)
SEQ ID NO: 12
Ala-Val-Ser-Glu-His-Gln-Leu-Leu-His-Asp-Lys-Gly-
Lys-Ser-Ile-Gln-Asp-Leu-Arg-Arg-Arg-Phe-Phe-Leu-
His-His-Leu-Ile-Ala-Glu-Ile-His-Thr-Ala-Tyr-Gly-
Phe-Gly-Gly;
(5)
SEQ ID NO: 13
Ala-Val-Ser-Glu-His-Gln-Leu-Leu-His-Asp-Lys-Gly-
Lys-Ser-Ile-Gln-Asp-Leu-Arg-Arg-Arg-Phe-Phe-Leu-
His-His-Leu-Ile-Aib-Glu-Ile-His-Thr-Ala-Tyr-Arg-
Phe-Gly-Gly;
(6)
SEQ ID NO: 14
Ala-Val-Ser-Glu-His-Gln-Leu-Ile-His-Asp-Lys-Gly-
Lys-Ser-Ile-Gln-Glu-Leu-Arg-Arg-Arg-Phe-Phe-Leu-
His-His-Leu-Ile-Aib-Glu-Ile-His-Thr-Ala-Tyr-Gly-
Phe-Gly-Gly;
(7)
SEQ ID NO: 15
Ala-Val-Ser-Glu-His-Gln-Leu-Ile-His-Asp-Lys-Gly-
Lys-Ser-Ile-Gln-Glu-Leu-Arg-Arg-Arg-Phe-Phe-Leu-
His-His-Leu-Leu-Ala-Glu-Ile-His-Thr-Ala-Tyr-Gly-
Phe-Gly-Gly.

13. The active polypeptide compound according to claim 1, further includes a compound obtained by chemically modifying the side chain groups of amino acids of the polypeptide compound; or

a coordination compound, a complex or a chelate formed by the polypeptide compound and a metal ion; or

a hydrate or a solvate formed by the polypeptide compound.

14. The active polypeptide compound according to embodiment 13, wherein the compound obtained by chemically modifying the side chain groups of amino acids of the polypeptide compound is a thioether or thioglycoside formed from a sulfydryl in a cysteine in the polypeptide compound, or a compound having a disulfide bond formed from a cysteine or a peptide comprising cysteine; or

an ester, an ether and a glycoside formed from a phenolic hydroxyl group of a tyrosine in the polypeptide compound; or

a compound prepared by substituting a benzene ring of a tyrosine or phenylalanine in the polypeptide compounds.

15. A pharmaceutical composition, comprising the active polypeptide compound according to any one of embodiments 1-14, and at least one of a pharmaceutically acceptable adjuvant, excipient, carrier and solvent thereof.

16. The pharmaceutical composition according to embodiment 15, comprising other therapeutic agents, which are selected from a drug that inhibits bone resorption, a drug that promotes ossification, a drug that promotes bone mineralization and a parathyroid hormone-related protein.

17. The pharmaceutical composition according to embodiment 16, wherein the drug that inhibits bone resorption includes calcitonin, diphosphonate, oestrogen, a selective oestrogen receptor regulator and isoflavone; the drug that promotes ossification includes fluoride, synthesized steroid, parathyroid hormone and parathyroid hormone-related protein; the drug that promotes bone mineralization includes a calcium agent, vitamin D and active vitamin D; and the parathyroid hormone-related protein is teriparatide or abaloptide.

18. Use of the compound according to any one of embodiments 1-4 and the pharmaceutical composition according to any one of embodiments 15-18 in the preparation of a medicament for preventing, treating or alleviating diseases or disorders related to osteogenic defects or bone mineral density decreasing, and the diseases include osteoporosis.

The above contents are further detailed descriptions of the present disclosure in combination with specific preferred embodiments, but it cannot be considered that the specific implementations of the present disclosure are limited to these descriptions. For one of ordinary skill in the art to which the present disclosure pertains, without deviating from the concept of the present disclosure, several simple deductions or replacements can also be made, which should all be regarded as belonging to the protection scope of the present disclosure.

1. An active polypeptide compound, which has a structure represented by following Formula (Ia) or Formula (Ib), or is a pharmaceutically acceptable salt thereof,
Y-ID-X Formula (Ia), or
X-ID-Y Formula (Ib),
wherein,
Y is a PTH/PTHrP receptor agonist or an osteoclast inhibitor;
ID is a peptide bond or a linker in the molecule, which links X to Y; and
X is an osteogenic growth peptide receptor agonist, a bone marrow mesenchymal stem cell irritant or a hematopoietic stem cell irritant.

2. The active polypeptide compound according to claim 1, wherein Y is M-CSF antagonist, RANKL inhibitor, RANKL antibody, MMP inhibitor, calcitonin, parathyroid hormone or parathyroid hormone-related protein.

3. The active polypeptide compound according to claim 1, wherein Y is a peptide chain having an amino acid sequence as shown in Formula (II):
A₁-Val-Ser-Glu-His-Gln-Leu-A₈-His-Asp-Lys-Gly-Lys-Ser-Ile-Gln-A₁₇-Leu-Arg-Arg-Arg-A₂₂-A₂₃-Leu-A₂₅-A₂₆-Leu-A₂₈-A₂₉-A₃₀-A₃₁-His-Thr-Ala Formula (II);
wherein, A₁ is Ala, Val, Leu or Ile;
A₈ is Leu or Ile;
A₁₇ is Asp or Glu;
A₂₂ is Glu, Asp or Phe;
A₂₃ is Leu, Ile or Phe;
A₂₅ is Glu, Asp or His;
A₂₆ is Lys, His or Arg;
A₂₈ is Leu, Ile or Val;
A₂₉ is Ala, (N-Me)Ala or Aib;
A₃₀ is Lys or Glu;
A₃₁ is Leu or Ile;
the amino terminal of the peptide chain Y is free or chemically modified, and the carboxyl terminal of the peptide chain Y is free or chemically modified.

4. The active polypeptide compound according to claim 1, wherein X is a hematopoietic stem cell irritant, a hematopoietic growth factor, a platelet colony-stimulating factor, a granulocyte colony-stimulating factor, erythropoietin, interleukin 3 or recombinant human interleukin 11.

5. The active polypeptide compound according to claim 1, wherein X is a peptide chain having an amino acid sequence as shown in Formula (IIIa) or Formula (IIIb):
Tyr-(Arg)ₘ-(Gly)ₙ-Phe-Gly-Gly Formula (IIIa)
Gly-Gly-Phe-(Gly)ₙ-(Arg)ₘ-Tyr Formula (IIIb);
wherein, m and n are independently 0, 1 or 2; and
the amino terminal of the peptide chain X is free or chemically modified, and the carboxyl terminal of the peptide chain X is free or chemically modified.

6. The active polypeptide compound according to claim 5, wherein X is a peptide chain consisting of 5-6 amino acids which has an amino acid sequence as shown in one of the following SEQ ID NO:1-SEQ ID NO:8:
(SEQ ID NO: 1)
Tyr-Gly-Phe-Gly-Gly
(SEQ ID NO: 2)
Tyr-Arg-Phe-Gly-Gly
(SEQ ID NO: 3)
Tyr-Arg-Gly-Phe-Gly-Gly
(SEQ ID NO: 4)
Tyr-Pro-Phe-Gly-Gly
(SEQ ID NO: 5)
Gly-Gly-Phe-Gly-Tyr
(SEQ ID NO: 6)
Gly-Gly-Phe-Arg-Tyr
(SEQ ID NO: 7)
Gly-Gly-Phe-Gly-Arg-Tyr
(SEQ ID NO: 8)
Gly-Gly-Phe-Pro-Tyr.

7. The active polypeptide compound according to claim 1, wherein ID is a linker between X and Y; the linker is an amino-substituted C₁₋₈ alkyl carboxylic acid, a polyethylene glycol polymer chain or a peptide segment consisting of 1-10 amino acids, and the amino acids in the peptide segment is selected from the group consisting of proline, arginine, alanine, threonine, glutamic acid, aspartic acid, lysine, glutamine, asparagine and glycine.

8. The active polypeptide compound according to claim 7, wherein the linker is one of the following linkers:
(1) (Gly-Ser)ₚ, wherein p is 1, 2, 3, 4 or 5;
(2) (Gly-Gly-Gly-Gly-Ser)ₜ, wherein t is 1, 2 or 3;
(3) Ala-Glu-Ala-Ala-Ala-Lys-Ala;
(4) 4-aminobutyric acid or 6-aminocaproic acid; and
(5) (PEG)_{q}, wherein q is 1, 2, 3, 4 or 5.

9. The active polypeptide compound according to claim 1, which has a structure as shown in Formula (IV), or is a pharmaceutically acceptable salt of the compound shown as in Formula (IV):
A₁-Val-Ser-Glu-His-Gln-Leu-A₈-His-Asp-Lys-Gly-Lys-Ser-Ile-Gln-A₁₇-Leu-Arg-Arg-Arg-A₂₂-A₂₃-Leu-A₂₅-A₂₆-Leu-A₂₈-A₂₉-A₃₀-A₃₁-His-Thr-Ala-A₃₅ Formula (IV),
wherein, A₁ is Ala, Val, Leu or Ile;
A₈ is Leu or Ile;
A₁₇ is Asp or Glu;
A₂₂ is Glu, Asp or Phe;
A₂₃ is Leu, Ile or Phe;
A₂₅ is Glu, Asp or His;
A₂₆ is Lys, His or Arg;
A₂₈ is Leu, Ile or Val;
A₂₉ is Ala, (N-Me)Ala or Aib;
A₃₀ is Lys or Glu;
A₃₁ is Leu or Ile; and
A₃₅ has a peptide chain of the amino acid sequence as shown in Formula (IIIa) or (IIIb):
Tyr-(Arg)ₘ-(Gly)ₙ-Phe-Gly-Gly Formula (IIIa),
Gly-Gly-Phe-(Gly)ₙ-(Arg)ₘ-Tyr Formula (IIIb),
wherein, m and n are independently 0, 1 or 2; and
the amino terminal of the amino acids shown by A₁ is free or chemically modified, and the carboxyl terminal of the peptide chain A₃₅ is free or chemically modified.

10. The active polypeptide compound according to claim 9, wherein the chemical modifications of the amino terminal include acylation, sulfonylation, alkylation and PEG modification; and the chemical modifications of the carboxyl terminal include amidation, sulfonylation and PEG modification.

11. The active polypeptide compound according to claim 10, wherein the chemical modification of amino terminal is acetylation, benzoylation or sulfonylation of amino; the alkylation of amino terminal is C₁₋₆ alkylation or aromatic alkylation; the chemical modification of carboxylic terminal is that the OH in the carboxyl is substituted by NH₂ or sulfamide, or the OH in the carboxyl links to the functionalized PEG molecule.

12. The active polypeptide compound according to claim 1, which is one of the compounds in the following SEQ ID NO:9-SEQ ID NO:15, or a pharmaceutically acceptable salt thereof:
(1)
SEQ ID NO: 9
Ala-Val-Ser-Glu-His-Gln-Leu-Leu-His-Asp-Lys-Gly-
Lys-Ser-Ile-Gln-Asp-Leu-Arg-Arg-Arg-Glu-Leu-Leu-
Glu-Lys-Leu-Leu-(N-Me)Ala-Lys-Leu-His-Thr-Ala-
Tyr-Gly-Phe-Gly-Gly;
(2)
SEQ ID NO: 10
Ala-Val-Ser-Glu-His-Gln-Leu-Leu-His-Asp-Lys-Gly-
Lys-Ser-Ile-Gln-Asp-Leu-Arg-Arg-Arg-Glu-Leu-Leu-
Glu-Lys-Leu-Leu-Aib-Lys-Leu-His-Thr-Ala-Tyr-Gly-
Phe-Gly-Gly;
(3)
SEQ ID NO: 11
Ala-Val-Ser-Glu-His-Gln-Leu-Leu-His-Asp-Lys-Gly-
Lys-Ser-Ile-Gln-Asp-Leu-Arg-Arg-Arg-Glu-Leu-Leu-
Glu-Lys-Leu-Leu-Ala-Lys-Leu-His-Thr-Ala-Tyr-Arg-
Gly-Phe-Gly-Gly;
(4)
SEQ ID NO: 12
Ala-Val-Ser-Glu-His-Gln-Leu-Leu-His-Asp-Lys-Gly-
Lys-Ser-Ile-Gln-Asp-Leu-Arg-Arg-Arg-Phe-Phe-Leu-
His-His-Leu-Ile-Ala-Glu-Ile-His-Thr-Ala-Tyr-Gly-
Phe-Gly-Gly;
(5)
SEQ ID NO: 13
Ala-Val-Ser-Glu-His-Gln-Leu-Leu-His-Asp-Lys-Gly-
Lys-Ser-Ile-Gln-Asp-Leu-Arg-Arg-Arg-Phe-Phe-Leu-
His-His-Leu-Ile-Aib-Glu-Ile-His-Thr-Ala-Tyr-Arg-
Phe-Gly-Gly;
(6)
SEQ ID NO: 14
Ala-Val-Ser-Glu-His-Gln-Leu-Ile-His-Asp-Lys-Gly-
Lys-Ser-Ile-Gln-Glu-Leu-Arg-Arg-Arg-Phe-Phe-Leu-
His-His-Leu-Ile-Aib-Glu-Ile-His-Thr-Ala-Tyr-Gly-
Phe-Gly-Gly;
(7)
SEQ ID NO: 15
Ala-Val-Ser-Glu-His-Gln-Leu-Ile-His-Asp-Lys-Gly-
Lys-Ser-Ile-Gln-Glu-Leu-Arg-Arg-Arg-Phe-Phe-Leu-
His-His-Leu-Leu-Ala-Glu-Ile-His-Thr-Ala-Tyr-Gly-
Phe-Gly-Gly.

13. The active polypeptide compound according to claim 1, further includes a compound obtained by chemically modifying the side chain groups of amino acids of the polypeptide compound; or
a coordination compound, a complex or a chelate formed by the polypeptide compound and a metal ion; or
a hydrate or a solvate formed by the polypeptide compound.

14. The active polypeptide compound according to claim 13, wherein the compound obtained by chemically modifying the side chain groups of amino acids of the polypeptide compound is a thioether or thioglycoside formed from a sulfydryl in a cysteine in the polypeptide compound, or a compound having a disulfide bond formed from a cysteine or a peptide comprising cysteine; or
an ester, an ether and a glycoside formed from a phenolic hydroxyl group of a tyrosine in the polypeptide compound; or
a compound prepared by substituting a benzene ring of a tyrosine or phenylalanine in the polypeptide compounds.

15. A pharmaceutical composition, comprising the active polypeptide compound according to claim 1, and at least one of a pharmaceutically acceptable adjuvant, excipient, carrier and solvent thereof.

16. The pharmaceutical composition according to claim 15, comprising other therapeutic agents, which are selected from a drug that inhibits bone resorption, a drug that promotes ossification, a drug that promotes bone mineralization and a parathyroid hormone-related protein.

17. The pharmaceutical composition according to claim 16, wherein the drug that inhibits bone resorption includes calcitonin, diphosphonate, oestrogen, a selective oestrogen receptor regulator and isoflavone; the drug that promotes ossification includes fluoride, synthesized steroid, parathyroid hormone and parathyroid hormone-related protein; the drug that promotes bone mineralization includes a calcium agent, vitamin D and active vitamin D; and the parathyroid hormone-related protein is teriparatide or abaloptide.

18. A method of preventing, treating or alleviating diseases or disorders related to osteogenic defects or bone mineral density decreasing, comprising administering a subject in need thereof the compound according to claim 1.

19. The method according to claim 18, wherein the disease is osteoporosis.
